(19) United States
(12) Patent Application Publication
(10) Pub. No.: US 2025/0095802 A1
(43) Pub. Date: Mar. 20, 2025

(54) SYSTEMS AND METHODS TO GENERATE PERSONAL DOCUMENTATION FROM AUDIO DATA

(71) Applicant: LABORATOIRE COEURWAY INC., Saguenay (CA)

(72) Inventors: Bastien GADOURY, Saguenay (CA); Jean-Phillipe GAGNON, Saguenay (CA); Mariane VAIL, Saguenay (CA); Pierre-Olivier GRAVEL, Saguenay (CA)

(73) Assignee: LABORATOIRE COEURWAY INC., Saguenay (CA)

(21) Appl. No.: 18/829,651

(22) Filed: Sep. 10, 2024

Related U.S. Application Data
(60) Provisional application No. 63/582,936, filed on Sep. 15, 2023.

(51) Int. Cl. G16H 10/60; G06F 40/174; G06F 40/186; G06F 40/197; G06F 40/40

(52) U.S. Cl. CPC G16H 10/60; G06F 40/186; G06F 40/197; G06F 40/40; G06F 40/174

(57) ABSTRACT

Systems and methods for generating personal documentation from audio data are disclosed. Meeting audio of a meeting between a person and a professional is received and transcribed, generating a transcription. A text string corresponding to a prompt is generated from the transcription, and is used to generate a document by a generative language model trained to take a string corresponding to the prompt as input and generate a string corresponding to the document as output. When the professional is a health professional and the person is a patient, the generated document can be a medical document such as a clinical note. Therefore, the systems and methods can be used as part of an electronic health records system.

### CROSS-REFERENCE TO RELATED APPLICATION

This application claims the benefit of, and priority to, U.S. Provisional Patent Application No. 63/582,936, filed Sep. 15, 2023, and entitled “SYSTEMS AND METHODS TO GENERATE PERSONAL DOCUMENTATION FROM AUDIO DATA”, the disclosure of which is hereby incorporated by reference in its entirety.

### TECHNICAL FIELD

The technical field relates to personal record management, and more specifically to systems and methods for generating personal documentation from audio data.

### BACKGROUND

Creating and maintaining records of interactions between professionals and persons is vital, and indeed legally required, for many professions. This can require scheduling time after each meeting to create and update documents, which is time-consuming and workflow-disruptive.

Recent advances in generative language models make it possible to produce documents automatically. Nonetheless, it is difficult to obtain documents that strictly adhere to format, structure and style requirements. In particular, tailoring the output of a generative language model to one's needs often require providing input prompts that include a massive amount of contextual information.

In applications that require processing large amounts of data, such as transcriptions of meeting audio, the high computational resource usage associated with running generative language models on large inputs and the limits generally imposed on input sizes, this becomes extremely problematic.

There remains a need for systems and methods that are able to leverage various tradeoffs that can be obtained when running a generation workflow in order to generate high quality personal documentation from audio data with limited computational resources.

### SUMMARY

According to an aspect, there is provided a system for generating personal documentation from audio data. The system includes at least one processing device and at least one memory for storing instructions executable by the at least one processing device. The instructions implement a number of modules. An audio acquisition module is configured to receive meeting audio of a meeting between a person and a professional and to store the meeting audio in the memory. A transcription module is configured to transcribe the meeting audio, generating a corresponding transcription and to store the transcription in the memory. A text generation module is configured to generate a document from at least one of the transcription and additional information. The text generation module includes a prompt engineering submodule configured to generate a text string corresponding to a prompt from the at least one of the transcription and the additional information, and a generative language model trained to take the text string as input and generate as output a continuation the text string, wherein the continuation text string corresponds to a continuation of the text string predicted by the generative language model, and wherein the continuation text string further corresponds to the document. A storage module is configured to securely store at least one of the meeting audio, the transcription, the additional information and the document in at least one of the memory and a distant persistent storage device. An assessment module is configured to receive an input from the professional corresponding to an assessment of a quality of the document. Responsive to the assessment being positive it generates one or more vectorial embeddings corresponding to at least one of the document and one or more portions of the document for use as future additional information to improve future document quality, and it causes the storage module to store the one or more vectorial embeddings in a vectorial database.

According to another aspect, there is provided a method for generating a medical document from audio data. The method includes authenticating a health professional, obtaining from storage a transcription of an audio recording associated with the health professional and a patient, generating a generative language model prompt from at least the transcription, and generating the medical document from the prompt.

According to a further aspect, there is provided a method for generating a new medical document from one or more medical documents. The method includes authenticating a health professional, obtaining from storage the one or more medical documents associated with the health professional and a patient, wherein the one or more medical documents include at least one of a meeting transcription, a medical note, and a professional assessment, generating a generative language model prompt from the one or more medical documents, and generating the new medical document from the prompt.

According to yet another aspect, a system for generating personal documentation from audio data is provided. The system includes at least one processing device and at least one memory for storing instructions executable by the at least one processing device, the instructions implementing modules, the modules comprising: an audio acquisition module configured to receive meeting audio of a meeting between a person and a professional and to store the meeting audio in the memory; a transcription module configured to transcribe the meeting audio, generating a transcription and to store the transcription in the memory; a text generation module configured to generate a document from at least the transcription, the text generation module comprising: a prompt engineering submodule configured to generate a text string corresponding to a prompt from at least the transcription, and a generative language model trained to take the text string as input and generate as output a continuation text string, wherein the continuation text string corresponds to a continuation of the text string predicted by the generative language model, and wherein the continuation text string further corresponds to the document; and a storage module configured to securely store at least one of the meeting audio, the transcription and the document in a persistent storage device.

According to yet a further aspect, a method for generating personal documentation from audio data is provided. The method includes receiving meeting audio of a meeting between a person and a professional; transcribing the meeting audio, generating a transcription; generating a text string corresponding to a prompt from at least the transcription; generating a document from the prompt, by a generative language model trained to take the text string as input and generate as output a continuation text string, wherein the continuation text string corresponds to a continuation of the text string predicted by the generative language model, and wherein the continuation text string further corresponds to the document; and securely storing at least one of the meeting audio, the transcription and the document in a distant persistent storage device.

According to yet another aspect, a computer readable memory having recorded thereon statements and instructions for execution by a computer is provided. The statements and instructions include receiving meeting audio of a meeting between a person and a professional; transcribing the meeting audio, generating a corresponding transcription; generating a text string corresponding to a prompt from at least the transcription; generating a document from the prompt, by a generative language model trained to take the text string as input and generate as output a continuation text string, wherein the continuation text string corresponds to a continuation of the text string predicted by the generative language model, and wherein the continuation text string further corresponds to the document; and securely storing at least one of the meeting audio, the transcription and the document in a distant persistent storage device.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment.

FIGS. 1A and 2B are schematics of systems for generating personal documentation from audio data, in accordance with two embodiments.

FIGS. 2A to 2F are flow charts of six methods for generating a medical document from audio data or for generating a new medical document from one or more medical documents, in accordance with six embodiments.

FIGS. 3A to 3X are views of a graphical user interface associated with an application for generating a medical document from audio data or for generating a new medical document from one or more medical documents, in accordance with an embodiment.

FIGS. 4A and 4B are plots of various performance metrics measured on different embodiments.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practised without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

One or more systems described herein may be implemented in computer program(s) executed on processing device(s), each comprising at least one processor, a data storage system (including volatile and/or non-volatile memory and/or storage elements), and optionally at least one input and/or output device. “Processing devices” encompass computers, servers and/or specialized electronic devices which receive, process and/or transmit data. As an example, “processing devices” can include processing means, such as microcontrollers, microprocessors, and/or CPUs, or be implemented on FPGAs. For example, and without limitation, a processing device may be a programmable logic unit, a mainframe computer, a server, a personal computer, a cloud-based program or system, a laptop, a personal data assistant, a cellular telephone, a smartphone, a wearable device, a tablet, a video game console or a portable video game device.

Each program is preferably implemented in a high-level programming and/or scripting language, for instance an imperative e.g., procedural or object-oriented, or a declarative e.g., functional or logic, language, to communicate with a computer system. However, a program can be implemented in assembly or machine language if desired. In any case, the language may be a compiled or an interpreted language. Each such computer program is preferably stored on a storage media or a device readable by a general or special purpose programmable computer for configuring and operating the computer when the storage media or device is read by the computer to perform the procedures described herein. In some embodiments, the system may be embedded within an operating system running on the programmable computer.

Furthermore, the system, processes and methods of the described embodiments are capable of being distributed in a computer program product comprising a computer readable medium that bears computer-usable instructions for one or more processors. The computer-usable instructions may also be in various forms including compiled and non-compiled code.

The processor(s) are used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory is, of course, chosen according to the desired use, whether it should retain instructions, or temporarily store, retain or update data. Steps of the proposed methods are implemented as software instructions and algorithms, stored in computer memory and executed by processors.

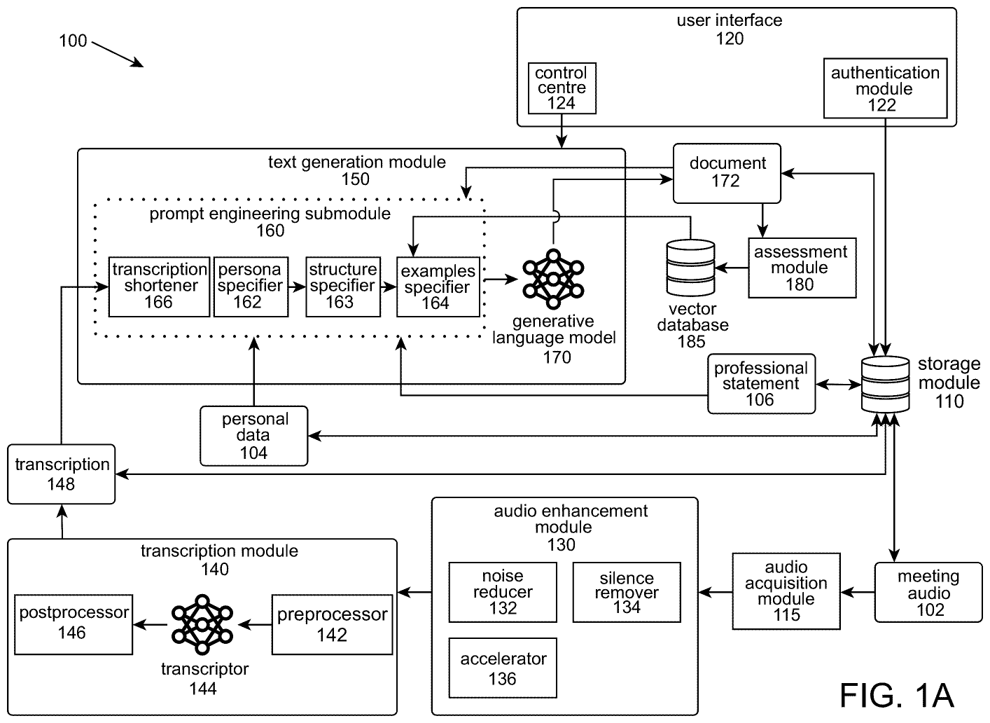

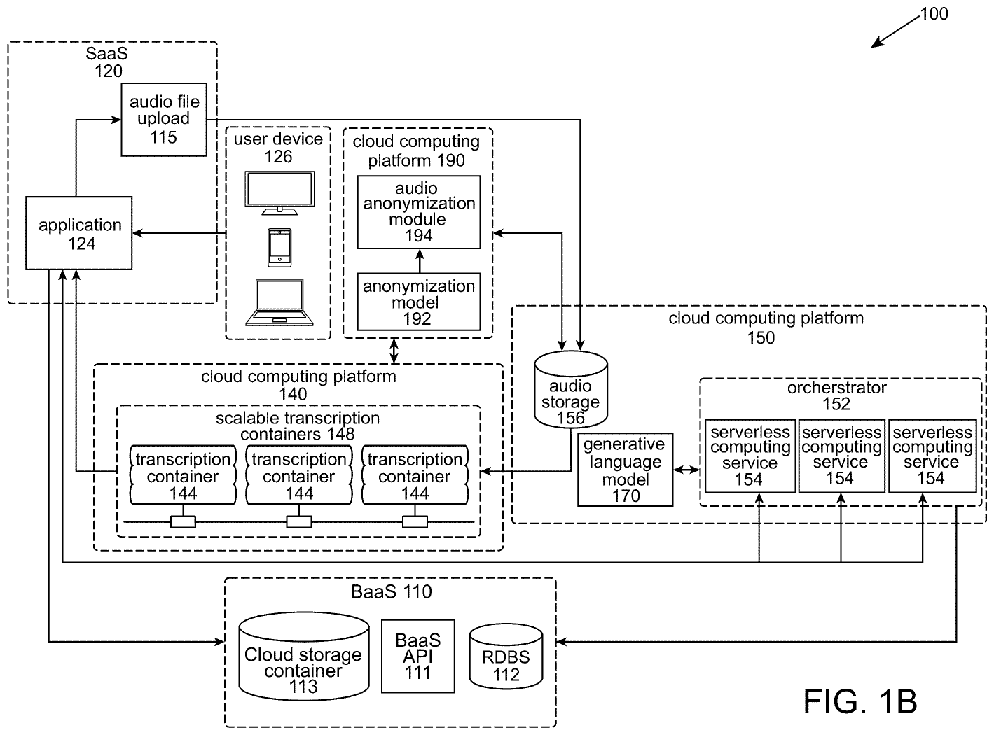

With reference to FIG. 1A, an exemplary system 100 for generating personal documentation from audio data is shown. Broadly described, the system 100 comprises modules for audio acquisition 115, enhancement 130 and transcription 140, for converting meeting audio 102 in a transcription 148. Upon receiving an instruction from a user interface 120 to create a personal document 172, the transcription 148 is passed to a text generation module 150 comprising a prompt engineering submodule 160 that uses the transcription and/or other information to create assemble a prompt to be used as the input of a generative language model 170. The output of the generative language model 170 is the document 172.

In some embodiments, the system 100 is provided within an electronic record system, for instance an electronic medical records system, an electronic health records system or an electronic docket system, such as for instance Medesync™, Myle™, Purkinje™, ProLaw™ or Clio™, or is configured to cooperate with an electronic record system. As an example, the system 100 can import personal data 104 from the electronic record system and can export any data or document 172 generated by the system 100 to the electronic record system.

The meeting audio 102 can include the recording of a meeting, for instance a consultation, between a person and a professional. Alternatively or additionally, the audio 102 can include the recording of a verbal professional statement 106 performed by the professional following the meeting with the person. In many embodiments, the professional works in a regulated occupation, for instance an occupation requiring the professional to maintain membership in a professional order, a regulatory college or some other form of a professional body. As an example, the professional can be a health professional such as an audiologist, a dentist, a dietician, a midwife, a nurse, an occupational therapist, a pharmacist, a physical therapist, a physician or a psychologist, in which case the person can be called a patient, and the personal document 172 can be called a medical document. Regulated occupations can be associated with stringent regulation regarding the form, storage and retention of records. As a further example, the professional can be an attorney, a lawyer, a notary or a solicitor, in which case the person can be called a client.

Personal data 104 can also be leveraged to inform document generation. Personal data 104 can for instance include sociodemographic information such as age, gender, education, employment and marital status. In the medical context, personal data 104 can be called patient data, and can include for instance signs, symptoms and medical history, including known diagnoses and known treatments.

The system 100 includes a storage module 110 configured to securely store all data related to professionals using the system 100 and to the persons the professionals are interacting with, as well as documents generated by the system. It can be used to store credentials for professionals to authenticate with the system, including for instance a username and a password, the hash of a password or the hash of a salted password, and/or biometric credentials for each professional. In some embodiments, some or all of the data stored by the storage module 110 are encrypted for secure storage. In some embodiments, symmetric-key cryptography can be used to encrypt data, wherein a key is used to encrypt and decrypt the data, for instance using any suitable cipher such as a stream cipher, e.g., RC4, or a block cipher, e.g., Advanced Encryption Standard (AES) or Camellia. As examples, the key can be owned and controlled by the system 100 and stored, e.g., in a secure vault, for automatic retrieval or use, or the key can be owned and controlled by the users of the system 100, or each user or group of users of the system 100 can have own and control their own key, such that encrypted data cannot be decrypted by other users of the system. In some embodiments, asymmetric-key cryptography, also called public-key cryptography, can be additionally or alternatively used to encrypt data, wherein a public key can for instance be used to encrypt data and the corresponding private key can be used to decrypt the data, using any suitable technique such as the Rivest-Shamir-Adleman (RSA) system or elliptic-curve cryptography (ECC). As an example, each user can own a key pair including a private key and a public key, the public key being stored, e.g., by the storage module 110 such that it can be used to encrypt data associated with the user, and they private key being stored on a device owned by the user such that it is impossible to decrypt the data without the device. In some embodiments, compression can be applied to data or files before encryption. It can be appreciated that applying compression before encryption makes it possible to maximize storage space savings, whereas applying compression after encryption would be less beneficial in terms of savings.

In some embodiments, the storage module 110 can be used to store audio data such as meeting audio 102. This can create a privacy issue, because voice is a sensitive biometric characteristic. In some embodiment, the system 100 includes an audio anonymization module configured to apply changes to the audio before storage. As an example, the audio anonymization module can apply voice modulation to voices in the audio data, for instance making modifications to one or more voice characteristics such as pitch, volume, tone and pace. In some embodiments, the characteristics to be modified and the modifications to be applied are selected randomly. In some embodiments, different modulations can be applied at different times in one recording. Known voice modulation algorithms can be applied. For instance, one algorithm only can be applied, or more than one algorithm can be applied to the same data, e.g., by using the output of a first algorithm as the input of a second algorithm, or different algorithms can be applied, alone or in combination, at different times in one recording. Preferably, the modulations are applied such that they make the voices unrecognizable and/or unusable as a biometric characteristic while not affecting the performance of the transcription module 140. In some embodiments, additionally or alternatively, an audio recording can be split in segments of uniform or different lengths and reassembled in a random order before storage to make abuse more difficult.

In some embodiments, the storage module 110 includes a database with a structure optimized to increase the performance of the storage module and/or to model complex relationships between the stored data. The database can for instance be implemented using a graph database such as an RDF store that can be manipulated or searched using a query language such as SPARQL or using a relational database management system (RDBMS) that can be manipulated or searched using a query language such as SQL. In some embodiments, the database can be hosted on a remote server provided with the system 100 or independently from the system 100, providing cloud storage. In some embodiments, the system 100 ensures that the cloud storage is configured such that all the data is stored in a suitable location having regard to existing regulations. As an example, a regulatory body may forbid personal being stored in a foreign country. A suitable backend can be provided to facilitate interactions between the modules of the system 100 and the storage module 110, for instance using a platform such as Firebase™ or Supabase™.

The system 100 includes an audio acquisition module 115, configured to receive the meeting audio 102. The audio acquisition module 115 can for instance include a sound recording device, such as a device including a microphone and an assembly configured to record an audio signal perceived through the microphone, for instance by polarizing a magnetic tape in proportion to the audio signal or by storing sound waves associated with the audio signal on any suitable storage device. In some embodiments, the meeting audio 102 can be acquired outside of the system 100, and the audio acquisition module 115 can have a function limited to obtaining the meeting audio 102 from a device on which it is stored. In some embodiments, the audio acquisition module 115 is configured, if necessary, to convert the recorded audio to a format suitable for use by the audio enhancement module 130 such as a bitstream file format, e.g., the Waveform Audio File Format or the Audio Interchange File Format. In some embodiments, the audio acquisition module is an application integrated within the user interface 120, for instance a web application or a native smartphone app that can be run by the professional on the Android™ or the iOS™ operating system and uses libraries made available by the system environment to record meeting audio 102 through the device's microphone. In some embodiments, the audio acquisition module 115 can be configured to extract an audio stream from a video recording, for instance a video recording performed by a videoconferencing application such as Zoom™, Google Meet™ or Microsoft Teams™, e.g., in the context of a teleconsultation.

The system 100 includes a user interface 120 configured to allow the professional to interact with the different modules of the system 100. The user interface can include an authentication module 122 configured to allow a professional to authenticate with the system 100, e.g., using authentication data stored by the storage module 110. Once the professional is authenticated, they can access data and documents stored in the storage module 110 that are relevant to them and access the different functionalities of the system 100, in particular the meeting creation and document generation functionalities through a control centre 124. In some embodiments, the user interface 120 can display a warning when a meeting was recorded but no corresponding note was entered after a configurable time period, e.g., 48 hours.

The system 100 includes an audio enhancement module 130 configured to perform different enhancements to the audio data, such as the meeting audio, before its transcription. These enhancements can for instance allow for a better quality of transcription and/or for less consumption of computational resources such as memory and processing time. The audio enhancement module 130 can include a noise reducer 132 configured to detect background noise in the meeting audio 102 and reduce or eliminate such background noise, to improve the transcription quality. The audio enhancement module 130 can include a silence remover 134 configured to detect moments of silence that were recorded during the meeting, e.g., moments when neither the professional nor the person is talking, and remove these moments from the meeting audio 102, thereby making the recording shorter and requiring less computational resources to process in subsequent steps. The audio enhancement module 130 can include an accelerator 136 configured to shorten the duration of the meeting audio 102 by accelerating its playback by a suitable factor, for instance making it 50%, 100% or 200% faster, thereby making the recording yet shorter and further requiring less computational resources to process in subsequent steps. It can be appreciated that the sound recording device and its microphone can be recognized automatically and that suitable audio enhancement parameters can be identified therefrom to be applied by the audio acquisition module 115 and/or the audio enhancement module 130.

The system 100 includes a transcription module 140 configured to transcribe the audio data. For instance, the transcription module can transcribe meeting audio 102, creating a transcription 148 for use by the text generation module 150. Additionally or alternatively, the transcription module can transcribe an audio recording of a professional statement, creating a written professional statement 106, also for use by the text generation module 150. The transcription module 140 can include a preprocessor 142 and a postprocessor 146. In some embodiments, the preprocessor is configured to split the audio data in many shorter audio segments, each segment being transcribed separately, thereby generating as many transcription segments as there are audio segments, and the postprocessor being configured to concatenate all the transcription segments in the right order to generate a single transcription corresponding to the totality of the audio data. This makes it possible to parallelize the transcription process, making the process faster. The transcription itself is performed by the transcriptor 144. It can be appreciated that any suitable architecture for computational speech recognition can be used in to implement the transcriptor 144. As an example, the transcriptor can first extract features from each audio segment, for instance by applying one or more of sampling, quantization, windowing, discrete Fourier transforming, mel filtering and logarithmizing techniques known to the art. The features can be used as input of a machine learning model trained for speech recognition. As an example only, the model can be a machine learning model such an attention-based encoder-decoder as described for instance in CHOROWSKI, Jan, BAHDANAU, Dzmitry, CHO, Kyunghyun, et al; End-to-end continuous speech recognition using attention-based recurrent NN: First results; arXiv preprint arXiv:1412.1602, 2014 and in CHAN, William, JAITLY, Navdeep, L E, Quoc, et al; Listen, attend and spell: A neural network for large vocabulary conversational speech recognition; In: 2016 IEEE international conference on acoustics, speech and signal processing (ICASSP); IEEE, 2016; p. 4960-4964, the entire disclosures of which are incorporated herein by reference, or a Connectionist Temporal Classifier, as described for instance in GRAVES, Alex, FERNÁNDEZ, Santiago, GOMEZ, Faustino, et al.; Connectionist temporal classification: labelling unsegmented sequence data with recurrent neural networks; In: Proceedings of the 23rd international conference on Machine learning; 2006; p. 369-376, the entire disclosure of which is incorporated herein by reference.

In some embodiments, the transcriptor 144 is configured to perform speaker diarization and/or recognition using any suitable approach such as an end-to-end approach as described for instance in FUJITA, Yusuke, KANDA, Naoyuki, HORIGUCHI, Shota, et al.; End-to-end neural speaker diarization with self-attention; In: 2019 IEEE Automatic Speech Recognition and Understanding Workshop (ASRU); IEEE, 2019; p. 296-303, the entire disclosure of which is incorporated herein by reference, and a voice activity detection approach as described for instance in MEDENNIKOV, Ivan, KORENEVSKY, Maxim, PRISYACH, Tatiana, et al.; Target-speaker voice activity detection: a novel approach for multi-speaker diarization in a dinner party scenario; arXiv preprint arXiv:2005.07272, 2020, the entire disclosure of which is incorporated herein by reference. A transcriptor 144 implementing speaker diarization and/or recognition can generate a transcription 148 that includes annotations such as an indication of the individual that is talking at each given time.

In some embodiments, the transcriptor 144 is configured to perform sentiment analysis using any suitable approach based on acoustic information, textual information obtained from the transcription, or a combination of both acoustic and textual information, as described for instance in LU, Zhiyun, CAO, Liangliang, ZHANG, Yu, et al; Speech sentiment analysis via pre-trained features from end-to-end ASR models; In: ICASSP 2020-2020 IEEE International Conference on Acoustics, Speech and Signal Processing (ICASSP); IEEE, 2020; p. 7149-7153, the entire disclosure of which is incorporated herein by reference. A transcriptor 144 implementing sentiment analysis can generate a transcription 148 that includes annotations such as an indication of a sentiment being conveyed by an individual in each given speech segment. In some embodiments, sentiment labelling can be ordinal and include labels such as “positive”, “negative” and “neutral”. In some embodiments, sentiment labelling can include labels, e.g., categorical or dimensional labels, from richer theories of emotion, as described for instance in AHMAD, Zishan, JINDAL, Raghav, EKBAL, Asif, et al.; Borrow from rich cousin: transfer learning for emotion detection using cross lingual embedding; Expert Systems with Applications, 2020, vol. 139, p. 112851, the entire disclosure of which is incorporated herein by reference.

The system 100 includes a text generation module 150. The transcription 148 or, in some embodiments, the annotated transcription output by the transcription module can be used as input to the text generation module 150. The text generation module 150 can additionally or alternatively rely on different inputs, such as a professional statement 106, which can be a written statement and/or the transcription of a verbal statement and personal data 104, e.g., patient data in medical applications. In some embodiments and for some applications, the text generation module 150 can additionally rely on embeddings retrieved from a vector database 185. Broadly described, the text generation module 150 includes a prompt engineering module 160 configured to create a suitable input for a generative language model 170, which is ultimately responsible for generating a document 172, which can for instance be a meeting summary, a meeting note, a personal recommendation, a filled form or a case history. It can be appreciated that other types of documents can be generated, and that different types of substantially similar documents can be known under different names, for instance between different fields. As an example, in health embodiments, the meeting note can be called a medical note, a clininal note, or a clinical meeting note.

The text generation module 150 includes a prompt engineering submodule 160. It can be appreciated that generative language models typically accept as input a vector, for instance a vector of integers, corresponding to tokens retrieved from a text string, e.g., natural language text. The text string used to create the input is typically called a “prompt”. Different techniques can be used to create a prompt that will, for a specific generative language model, produce the desired output, as described for instance in BROWN, Tom, MANN, Benjamin, RYDER, Nick, et al.; Language models are few-shot learners; Advances in neural information processing systems, 2020, vol. 33, p. 1877-1901, the entire disclosure of which is incorporated herein by reference. This can be referred to as “prompt engineering”. The text string corresponding to the prompt is typically mapped to the vector passed as input to the generative language model by an injective function implemented by a tokenizer, thereby the text string, the prompt and the input vector can be described as equivalent. In the present disclosure, therefore, “input text”, “input vector” and “prompt” will be used interchangedly.

Some generative language models provide an API that allows for passing one or more messages of different types, which are ultimately used as a prompt to create the input of the generative language. As an example, the OpenAI™ ChatGPT™ API can accept as input one or more messages according to three different roles: “system”, “user” and “assistant”, which can be leveraged as will be described in more details below.

The prompt engineering submodule 160 can include a persona specifier 162. The persona specifier 162 is configured to generate a portion of the prompt that set up a context and/or a behaviour of the generative language model. The portion of prompt can for instance include language directed at directing the generative language model to adopt a persona associated with a given profession and/or directing the model to produce a certain, desired type of document. In a first example embodiment, the professional may be a physical therapist. The physical therapist can use the control centre 124 to request that a Subjective, Objective, Assessment and Plan (SOAP) note be generated, for instance from the transcription 148 of a meeting with a patient and/or from a document 172 stored in by the storage module 110 corresponding to a summary of said meeting, and optionally a professional statement 106. In this first example, the persona specifier 162 could generate a portion of prompt stating: “You are a physical therapist that has just met with a patient for the first time and must write a SOAP note.” In a second example embodiment, the professional may be a patent attorney. The patent attorney can use the control centre 124 to request that a client intake note be generated, for instance from the transcription 148 of a meeting with an inventor and/or from a document 172 stored in by the storage module 110 corresponding to a summary of said meeting, and optionally a professional statement 106. In this second example, the persona specifier 162 could generate a portion of prompt stating: “You are a patent attorney that has just met with an inventor for the first time and must write an intake note.” In some embodiments, the persona specifier 162 can be configured to directing the generative language model to adopt a persona associated with a given profession which might be different from that of the professional, for instance a professional transcriptor, also called a scribe. As an example, if the professional is a health professional, a lawyer, or a patent attorney, the persona specifier may direct the model to adopt, respectively, the persona of a medical scribe, of a legal scribe or of an intellectual property scribe, i.e., a suitable documentation specialist.

It can be appreciated that, in adopting a persona associated with a given profession, the model can be implictly directed to adopt a suitable professional style and a professional tone associated with said profession and to address an audience composed of a certain type of professionals, e.g., peers of the professional defined by the persona, i.e., professionals of the type or of a related type. For instance, by adopting the persona of a health professional and/or of a medical scribe, the model can be directed to generate documents addressed to an audience composed of their peers, such as health professionals. These style, tone and audience attributes can be called derivative attributes of the persona. It can be appreciated that other derivative attributes are possible, including for instance the use of terminology and jargon, a level of attention to detail, concision, regulatory compliance, consistency, expertise and cultural sensitivity. In some embodiments, some or all derivative attributes of the persona can be specified explicitely by the personal specifier for inclusion in the prompt. As an example, the style associated with the persona can be prescribed in minute details, including for instance instructions to use a specific writing style and/or a certain jargon, and/or instructions to use specific, listed abreviations, symbols and/or expressions.

The prompt engineering submodule 160 can include a structure specifier 163. The structure specifier 163 is configured to generate a portion of the prompt that set up the desired text structure, for instance associated with the type of profession and the type document desired. This indication of text structure can include textual instructions for the generative language model to prepare a desired document type, e.g., Subjective, Objective, Assessment, Plan, and Interventions (SOAPI), the Provocative/Palliative, Quality/Quantity, Region/Radiation, Severity, Timing/Treatment and Understanding (PQRSTU) or the Data, Intervention and Results (DIR) type or a derivative thereof, and to observe a desired text structure comprising a plurality of document sections, the indication of text structure selected based on the desired document type from a set comprising predetermined indications of text structure. The provided indication can include low-level indications and high-level indications. As an example, a low-level indication can include specific instructions related to the sections or each section that should be included in the generated document. As an example, a high-level indication can include a more general description of the type of document that ought to be generated.

In some generative language model APIs, the prompt portion generated by the persona specified 162 and/or by the structure specifier can correspond to a message. As an example only, in ChatGPT™, the message may be represented in JSON as the following “system” message: {“role”: “system”, “content”: “You are a physical therapist that has just met with a patient for the first time and must write a SOAP note.”}.

The prompt engineering submodule 160 can include an examples specifier 164. The examples specifier 164 is configured to generate one or more portions of the prompt that provide examples of outputs expected by the generative language model. In the example embodiments described above, the examples specifier 164 can for instance generate one or more portions of prompt including examples of complete or partial SOAP notes or intake notes that have been judged to be of high quality. In some embodiments, at least one of these examples is predefined and provided with the system 100. In some embodiments, each example of output can also include a corresponding example of input or portion of an input that would have generated said output. As an example, an example can include the transcription of a real or fictitious meeting between a professional and a person and the document produced as a result of this meeting.

In some embodiments, at least one of these examples is provided by a user of the system 100, for instance through configuration files or through the user interface 120, for instance defining a preferred note structure for one or more users of the system 100. In some embodiments, at least one of these examples is retrieved from a vector database 185, as will be described in further details below. In some embodiments, at least one of these examples is defined by a user using a generation template builder tool provided by the user interface 120, which makes it possible for a user to create and modify document structures, for instance by adding, removing or modifying fields from a structure. In some embodiments, the generation template builder tool uses the generative language model 170 and/or embeddings stored in the vectorial database 185 to assist a user in creating or modifying a document structure interactively. As an example, the generative language model 170 and/or embeddings stored in the vectorial database 185 can be leveraged to suggest to a user the inclusion of certain fields that are found in other documents. In some embodiments, at least one of these examples is automatically selected from documents previously generated following a request of the professional using the system that have been assessed as particularly satisfactory by that same professional, in order to increase the subjective quality of the generated document by more closely matching the professional's personal preferences. In some embodiments, one or more types of examples as described above are used. In some embodiments, at least one of the examples is provided as a standalone example with no provided context. In some embodiments, at least one of the examples is provided as a standalone example with no provided with a context, for instance a context corresponding to a prompt or a part of a prompt that has or could have been used to generate the example. In some embodiments, alternative or additional approaches make it possible to personalize the output of the professional for each professional or for a group of professionals. As an example, professionals can opt to use personalized document sections, which can be aggregated with the prompt as part of a low-level indication of a text structure provided by the text structure specifier 163.

In some generative language model APIs, the one or more prompt portions generated by the examples specifier 164 can correspond to one or more messages. As an example only, in ChatGPT™, messages may be represented in JSON as the following array of alternated “user” and “assistant” message, respectively indicating a prompt that could have been used to generate the example and the example as would have been generated by the generative language model: [{“role”: “user”, “content”: “Generate a SOAP note for the patient meeting summarized hereafter: [ . . . ]”}, {“role”: “assistant”, “content”: “Patient consults for an ankle pain. Patient reports that the pain appeared 48 hours ago. [. . . ]”}].

The prompt engineering submodule 160 can include a transcript shortener 166. As can be appreciated from the amount and types of information that can be included in the prompt, the input of the generative language model 170 can be large. In particular, in many embodiments, the transcription 148 will be included in the prompt. The transcript can be large. As an example, if the professional and the person talk for 15 minutes at a rate between 125 and 150 words per minute, the transcript of their meeting will include between 1,875 and 2,250 words, which depending on the language and the tokenizer used may translate into 2,500 to 4,500 tokens. The more tokens are provided as input to a generative language model, the more memory and processing time are required to generate the output. Moreover, certain generative language models have a limit on the input length, such as 2,049, 4,097 or 16,385 tokens. Therefore, being able to limit the number of tokens required by the different portions of a prompt can be effective in both ensuring better computational performance of the generative language model and in improving the quality of its output, by providing more information in input using the same or a lesser number of tokens. Additionally or alternatively, it can be desirable to limit the size of the output of the language model, for instance to reduce the usage of computational resources and/or because the language model may have a limit on the output length and/or on the combined input and output length.

It can be appreciated that tokenizers use encodings that are frequently trained on the corpus that is subsequently used to train the generative language model. Tokenizer encodings are trained to optimize the model's performance with respect to its training corpus, which usually results in tokenizer encodings substantially acting as entropy encodings. Many of the most prominent generative language models are trained on a corpus that is mostly composed on English text. As an example, GPT™ 3 is said to have been trained on a corpus containing more than 90% of English text. It therefore follows that tokenizers will tend to encode non-English text less efficiently, making transcription shortening even more important to performance when the transcription 148 is not in English.

Different approaches can be used to limit the number of tokens used in a prompt, in particular for the transcription 148. One approach includes removing certain words that bear a relatively limited contribution to the meaning of an utterance. As an example, words that are part of certain word classes can be deemed to contribute less to the meaning of an utterance. Another approach includes removing certain predetermined characters that add disproportionately to the token count and can be removed with no or limited adverse effects on the quality of the generative language model output, optionally replacing them with other characters. As an example, letters with diacritics such as “ä”, “č” or “é” in the tokenizers associated to numerous generative language models each correspond to one token, and can often be replaced with corresponding letters such as “a”, “c” or “e” with limited adverse effects. As another example, punctuation signs such “'”, “,” or “.” also often each count as one token, and can often be removed with limited adverse effects. Another approach includes converting the transcription, a part of the transcription, the prompt or a part of the prompt to a predetermined alternative orthographic and/or syntactic system. As an example, the traditional French spelling “oignon” (onion) can be advantageously replaced with the reformed spelling “ognon” to save one token in certain tokenizers such as those using the “cl 100k_base” encoding from GPT™. As another example, a phonemic orthographic system, wherein one phoneme corresponds to exactly one letter and one letter corresponds to exactly one phoneme, or a phonetic orthographic system, wherein one sound corresponds to exactly one letter and one letter corresponds to exactly one sound, can be used instead of a standard orthographic system. For instance, replacing the vowel cluster “eau” with the vowel “o” in French, which correspond to the same phoneme and to the same sound, can save one token in certain tokenizers. As yet another example, syntactic transformations that have the potential to reduce the number of tokens can be applied, including for instance in French replacing the “ne . . . pas” negations with simple “pas” negations, saving one token in certain tokenizers, and/or replacing “est-ce que” questions with subject-verb inversion questions, saving three tokens in certain tokenizers. Another approach includes replacing certain predetermined words with corresponding predetermined abbreviations and/or synonyms that are known to require fewer tokens in the target tokenizer. As an example, replacing the French word “professionnel” with its shorter synonym “pro” saves two tokens in certain tokenizers. Another approach includes translating the transcription, a part of the transcription, the prompt or a part of the prompt in a different language, for which the tokenizer is better optimized. Depending on the embodiments, one, some, all or none of these approaches can be used to limit the number of tokens.

The efficiency of these approaches can be verified empirically. As an example, Douglas Adams famously wrote: “There is no problem so complicated that you can't find a very simple answer to it if you look at it right.” When tokenized with GPT™'s “cl100k_base” encoding, this sentence corresponds to 24 tokens. The French translation of this sentence is: “Il n'est pas de problème si compliqué qu'on ne puisse y apporter une solution très simple si on le prend par le bon bout.” The same encoding produces 39 tokens for the French version, i.e., 62.5% more. Replacing the “é” and “è” characters with “e” in French removes 4 tokens. Removing the punctuation marks remove 3 additional tokens. Removing the determiners and pronouns remove 8 tokens. In total, when all of these approaches are applied, the French sentence has 25 tokens, which would substantially improve the performance of the language model, but which is still one more than the English sentence. Simply translating the French sentence to English removes 15 tokens. Subsequently removing punctuation signs, determiners and pronouns from the English sentence remove 6 more tokens. Therefore, by combining some of the approaches described above, it is possible to more than halve the number of tokens of one example sentence, from 39 to 18. As a further example, the same sentence in Korean would require 87 tokens. Therefore, translating the Korean sentence in English and removing punctuation signs, determiners and pronouns would remove 69 tokens, thereby dividing the number of tokens by nearly 5.

In some embodiments, the prompt engineering submodule 160 is configured to invoke the language model more than one time to generate portions of the document. In other words, the text generation module 150 can be configured to generate a plurality of intermediary documents from a corresponding plurality of prompts, each prompt of the plurality based on at least the transcription, an indication of a document section and an indication of at least one example generated by an examples specifier, the indication of at least one example comprising textual instructions for the generative language model to rely on the at least one example in generating the document, each of the at least one example comprising at least an example transcription and an example section, each of the at least one example being selected from a set comprising predetermined examples, the plurality of intermediary documents corresponding to a plurality of document sections, then to generate the document by concatenating the plurality of intermediary documents. This can advantageously cause the output size to be shorter, because only a portion of the document is generated at each invocation, and can also advantageously cause the input size to be shorter, because examples of only a portion of the desired document is aggregated with the prompt at each invocation. In addition to reducing the usage of computational resources and/or of allowing the production of longer documents, this approach causes splitting the document generation task in a number of simpler sub-tasks, which can result in higher quality documents. In some embodiments, the document resulting from the concatenation of the intermediary documents can be used as part of the prompt of an addition, final invocation of the language model, including instructions for the model to ensure consistency, for instance by enforcing stylistic consistency and/or by avoiding contradictions or repetitions of information. The additional invocation can provide a quality insurance, by ensuring the document includes complete and precise information, that the relevant information from the transcription is well understood and reflected, without errors or omissions in the document, and are conveyed clearly and without ambiguity.

In some embodiments, the prompt engineering submodule 160 includes additional or alternative text in the prompt beyond the transcription 148 and/or the shortened transcription, the professional statement 106, and the output of the persona specifier 162, of the structure specifier 163 and of the examples specifier 164.

In particular, the prompt engineering submodule 160 can include documents previously generated by the text generation module 150 in a prompt. For instance, this approach can advantageously be used to generate intermediary documents that correspond to a compressed version of an input prompt and/or document, in the sense that they contain substantially the same information but use fewer tokens, and/or have a higher information/token ratio. As an example, in some embodiments, the text generation module 150 can be used, in a first time, to generate a summary of a meeting from a meeting audio 102, and, in a second time, to generate a note from the summary and from the professional statement 106. This two-stage generation process can result in better computational performance and/or better note quality than a one-stage generation process from an extremely large prompt including both a complete transcription 148 of the meeting audio 102 and the professional statement 106 at the same time. In some embodiments, a multiple-stage generation process can be used. In some embodiments, advantageously, the same generative language model 170 is used for each stage of a two-stage or multiple-stage generation process.

In some embodiments, the professional can input or download personal data 104 for storage by the storage module 110, or access personal data 104 through any suitable database. The prompt engineering submodule 160 can retrieve this personal data 104, create a textual representation corresponding to it, and include it in the prompt. As an example, in a medical embodiment, the prompt engineering submodule 160 may retrieve structured patient data such that can for instance be represented in JSON as {“gender”: 0, “day of birth”: 19, “month of birth”: 9, “year of birth”: 1993, “marital status”: 1, “education”: 3, “employment status”: 2, “occupation”: “programmer”, “diagnoses”: [{“diagnosis”: “diabetes mellitus”, “day”: 5, “month”: 2, “year”: 2020}], “treatments”: [{“medication”: “metformin”, “dosage”: 850, “dosage unit”: “mg”, “dosage frequency”: “BID”, “route”: “oral”}], “tests”: [{“day”: 15, “month”: 6, “year”: 2023, “variables”: [{“name”: “A1C”, “value”: “5.5”, “unit”: “%”}]}, {“day”: 13, “month”: 6, “year”: 2022, “variables”: [{“name”: “A1C”, “value”: “6.3”, “unit”: “%”}]}]} and, using any suitable method such as rule-based generation, generate a portion of a prompt that reads “Patient is a 30 year old married male with an undergraduate degree, currently employed full-time as a programmer. Patient has been followed for diabetes mellitus for 3 years and takes metformin 850 mg BID PO. Patient had decreasing A1C at 5.5% on 15 Jun. 2023.”

In some embodiments, the professional can use the user interface 120 to input additional information during a meeting, and the corresponding text can be passed on to the prompt engineering submodule 160 for inclusion within the prompt.

In some embodiments, the prompt engineering submodule 160 is configured to include instructions associated with a regulatory body of the system user's profession in the prompt. As an example, the regulatory body for a given profession may provide record-keeping prescriptions including specific structures that is to be used for certain types of notes or specific information that must be included in certain types of notes. In some embodiments, at least some of the prescriptions are obtained by an administrator of the system 100 and stored, for instance in a configuration file or by the storage module 110. In some embodiments, at least some of the prescriptions are obtained automatically by the system 100, e.g., by scraping the website of a regulatory body and applying known natural language processing methods to retrieve prescriptions from the website content. In some embodiments, the text of each relevant prescription is included as is within the prompt. In some embodiments, the text of each relevant prescription is included with an optional prefix and/or suffix within the prompt. As an example, the text string “When preparing the note, make sure to observe the following prescription: ‘[prescription text]’” can be concatenated with the prompt. In some embodiments, a text string explaining each prescription with a structure optimized for the generative language model can be generated manually or automatically and stored in the system 100 for inclusion within the prompt when relevant.

In some embodiments, “prompt chaining” is used in addition or as an alternative to prompt engineering. A first prompt is generated and used as input to the generative language model 170, which outputs text that is included in a second prompt to be used by the same or by a different generative language model 170. This process can be repeated as needed.

The system 100 includes a generative language model 170 trained to accept an input corresponding to a prompt and to predict a continuation of the prompt. That is, given an input corresponding to an “input text string” containing text, e.g., in a natural language, the generative language model 170 outputs an “output text string”, which is a prediction of the text that is expected to occur after the input text string in order to create a complete text that is similar to text that was observed by the language model during training. More specifically, a generative language model 170 typically predicts one character, one word or one token from the vector corresponding to the input string, then appends the predicted character, word or token to the vector and uses the extended vector to predict a subsequent character, word or token inductively. It can be appreciated that different types of models can be used to implement a generative language model, for instance probabilistic models such as n-gram language models, as described for instance in SHANNON, Claude Elwood; A mathematical theory of communication; The Bell system technical journal, 1948, vol. 27, no 3, p. 379-423, the entire disclosure of which is incorporated herein by reference. Neural networks, in particular recurrent neural networks and their derivatives, are particularly suitable to implement generative language models. Neural network-implemented generative language models are a type of neural language model. It is understood that the neural networks can be implemented using computer hardware elements, computer software elements or a combination thereof. Accordingly, the neural networks described herein can be referred to as being computer-implemented. Various computationally intensive tasks of the neural network can be carried out on one or more processors (central processing units and/or graphical processing units) of one or more programmable computers. For example, and without limitation, the programmable computer may be a programmable logic unit, a mainframe computer, server, personal computer, cloud-based program or system, laptop, personal data assistant, cellular telephone, smartphone, wearable device, tablet device, virtual reality device, smart display devices such as a smart TV, set-top box, video game console, or portable video game device, among others.

In some embodiments, the generative language model 170 is an autoregressive model, for instance a recurrent neural network (RNN), including stacked RNNs, bidirectional RNNs, long short-term memory networks as described for instance in HOCHREITER, Sepp et SCHMIDHUBER, Jürgen; Long short-term memory; Neural computation, 1997, vol. 9, no 8, p. 1735-1780, the entire disclosure of which is incorporated herein by reference, RNNs with gated recurrent units as described for instance in CHO, Kyunghyun, VAN MERRIËNBOER, Bart, GULCEHRE, Caglar, et al.; Learning phrase representations using RNN encoder-decoder for statistical machine translation; arXiv preprint arXiv:1406.1078, 2014, the entire disclosure of which is incorporated herein by reference, and RNNs of any type implementing an attention mechanism such as the dot-product attention, or a combination of neural networks including an RNN, for instance as a decoder, the whole as described for instance in SUTSKEVER, Ilya, MARTENS, James, et HINTON, Geoffrey E; Generating text with recurrent neural networks; In: Proceedings of the 28th international conference on machine learning (ICML-11); 2011; p. 1017-1024, the entire disclosure of which is incorporated herein by reference.

In some embodiments, the generative language model 170 is a transformer model, i.e., a neural network using one or more self-attention layers, for instance a an encoder-decoder transformer model, as described for instance in RAFFEL, Colin, SHAZEER, Noam, ROBERTS, Adam, et al.; Exploring the limits of transfer learning with a unified text-to-text transformer; The Journal of Machine Learning Research, 2020, vol. 21, no 1, p. 5485-5551, the entire disclosure of which is incorporated herein by reference, or a decoder transformer model, as described for instance in VASWANI, Ashish, SHAZEER, Noam, PARMAR, Niki, et al.; Attention is all you need; Advances in neural information processing systems, 2017, vol. 30, the entire disclosure of which is incorporated herein by reference. In some embodiments, the transformer model users as generative language model 170 is a pretrained model, as described for instance in RADFORD, Alec, NARASIMHAN, Karthik, SALIMANS, Tim, et al.; Improving language understanding by generative pre-training; 2018, and in DEVLIN, Jacob, CHANG, Ming-Wei, LEE, Kenton, et al.; BERT: Pre-training of deep bidirectional transformers for language understanding; arXiv preprint arXiv:1810.04805, 2018, the entire disclosures of which are incorporated herein by reference, and in BROWN, Tom, MANN, Benjamin, RYDER, Nick, et al., cited above. A pretrained generative language model is “trained” (or pretrained) from a very large quantity of text of different natures gathered from different sources, e.g., ca. 570 GB of text corresponding to ca. 499,000,000,000 tokens, to obtain what can be called a foundational model suitable to be adapted to a specific task by a process known as “fine-tuning”, as described for instance in OPENAI; Introducing ChatGPT [online]; [Accessed 13 Sep. 2023]; Available from: https://openai.com/blog/chatgpt, the entire disclosure of which is incorporated herein by reference. The use of a very large quantity of text makes it possible to train large language models, i.e., transformer neural networks with a large number of parameters, e.g., ca. 175,000,000,000.

In some embodiments, the generative language model 170 is an off-the-shelf fine-tuned generative language model, such as ChatGPT™. In some embodiments, the generative language model 170 is a custom pretrained generative language model or an off-the-shelf pretrained generative language model, such as GPT™ or BERT™, fine-tuned with a corpus of documents specific to the system 100, the profession of the system users and/or the types of documents 172 to be generated. In some embodiments, the generative language model 170 is fine-tuned with a number of pairs, each pair composed of one prompt and a corresponding document 172 that has been either hand-crafted by a professional, or generated, assessed positively and/or manually improved by a professional. In some embodiments, a distinct generative language model 170 is fine-tuned for each user of the system 100, for instance on documents that one given user has assessed positively and/or improved, yielding a number of personalized generative language models that are adapted to the personal style and preferences of each user. It can be appreciated that any desired level of granularity can be obtained in the personalization of the generative language models. As an example, an embodiment of system 100 could include one generative language model for each type of professional and/or for each type of document. In yet other embodiments, the generative language model 170 can be trained directly and without pretraining on a suitable corpus, for instance a corpus containing texts composed of a prompt and a document concatenated to the prompt. In some embodiments, when training or fine-tuning a generative language model 170 based on prompts and documents, wherein all or most of the prompts include a common substring, the substring is omitted from the fine-tuning corpus. This can result in the fine-tuned language model behaving as if the common substring was included in the prompt without having to actually include it in the prompt, reducing the size of the input without affecting the quality of the output, resulting in improved computational performances of the generative language model 170.

Once a document 172 has been generated by the text generation module 150, it can be stored by the storage module 110. It can additionally or alternatively be passed on to an assessment module 180. The assessment module 180 interacts with the user interface 120 to display the document 172 to the user and accept different types of user inputs. In some embodiments, the user can provide an assessment of the quality of the document 172, for instance a binary assessment, e.g. good or bad, an unidimensional assessment, e.g. using a Likert scale, or a multidimensional assessment, e.g., using more than one Likert scale, for instance a first scale indicating an assessment of the quality of the language in the document 172 and a second scale indicating an assessment of the compliance of the document 172 with the specific features of the type of document that was requested. The assessment of a document 172 can be stored along with the document 172 by the storage module 110. In some embodiments, the user can make modifications to the document, for instance to correct errors or inconsistencies, to remove superfluous text, to add missing text and/or to adapt the document to the preferences of the user. The modified document can be stored along with the original document 172 or instead of the original document 172 by the storage module 110. Documents that have been modified and/or have been assessed positively can advantageously be used, for instance by the examples specifier 164 or in fine-tuning the generative language model 170, to improve the performance of the text generation module 150. In some embodiments, all or some of the elements that have been modified or added by a user are recorded by the storage module 110 and are included within subsequent prompts for the same user and/or for the same document type.

In some embodiments, the system 100 includes a vector database 185. In some embodiments, the vector database 185 can be implemented by the storage module 110. In other embodiments, the vector database 185 corresponds to a different storage optimized for the storage and retrieval of vectors. In some embodiments, an off-the-shelf vector database such as Chroma™, Milvus™, Pinecone™, Qdrant™, Redis™, Typesense™, Weaviate™ or Zilliz™. The vector database is used to store persistently vector embeddings corresponding to documents, in particular documents that have been assessed positively and/or that have been modified, and/or portions of documents. In some embodiments, the personal data 104 of the person associated with a document 172 or a portion of a document stored in the vector database 185 is stored alongside the document or portion of document.

A vector embedding can be any suitable vectorial representation of the text of a document or a portion of a document. As an example, the same tokenizer used in the text generation module 150 or a different tokenizer can be used to obtain a vector, e.g., a fixed-dimensionality, zero-padded, integer vector corresponding to the tokens of the text, corresponding to one possible vectorial embedding of the text. As another example, the text can be provided as input to the same neural generative language model 170 used in the text generation module 150 or to another neural generative language model, to retrieve the output of a specific layer of the neural model, for instance the output of the first layer and/or the output of an embedding layer, in which case the vector embedding is more accurately described as a tensor embedding, the tensor shape depending on the structure of the model. As a further example, the text can be provided as input to an embedding model, i.e., a model specifically trained to map its input to a vector, such as variational autoencoder as described for instance in KINGMA, Diederik P. et WELLING, Max; Auto-encoding variational Bayes; arXiv preprint arXiv:1312.6114, 2013, the entire disclosure of which is incorporated herein by reference. The vector output by the embedding model corresponds to another possible vectorial embedding of the text, for instance a fixed-dimensionality vector of real numbers. As an example, the vector can have 1536 dimensions and be normalized so to a length of 1.

The benefit of storing documents or portions of documents as vectors rather than as text is that it makes retrieving relevant documents or portions of documents very inexpensive computationally by computing a similarity measure, e.g., a distance, between the vectorial embeddings, for instance a Euclidian distance or a cosine similarity. In some embodiments, the prompt generated by the prompt engineering submodule 160 can be converted to a corresponding prompt vectorial embedding, using the same approach used to convert the documents and portions of documents stored in the vector database 185, and this prompt vectorial embedding can be sent to the vector database 185 to retrieve a preconfigured number of most similar stored vectorial embeddings. In some embodiments, alternatively, the vectorial embeddings corresponding to personal data closest to the personal data 104 of the person for which a document is being generated can be retrieved. As an example, the stored personal data and the personal data 104 of the person can be converted to vectors, and the vectorial embeddings corresponding to the personal data vectors stored in the vector database 185 closest to the personal data 104 vector can be retrieved. In some embodiments, vectorial embeddings are retrieved based on an aggregation of their distance with the prompt vectorial embedding and the distance of their associated personal data vector with the personal data 104 vector. The aggregation can for instance be a sum, a mean or a weighted average using configurable weights. The retrieved vectorial embeddings or the corresponding text can then be concatenated with the prompt by the examples specifier 164.

With reference to FIG. 1B, an exemplary system 100 for generating personal documentation from audio data is shown. Broadly described, the system 100 includes a number of remote cloud infrastructures each implementing different parts of the system. As shown, exemplary system 100 includes an application server for serving the application as Software-as-a-Service (SaaS) 120 to user devices 126, a cloud infrastructure 140 implementing scalable transcription, a cloud infrastructure 130 implementing text generation, a cloud infrastructure 110 implementing storage, and a cloud infrastructure 190 implementing anonymization. The remote cloud infrastructures can include services best described as Infrastructure-as-a-Service (Iaas), as Platform-as-a-service (PaaS), as Backend-as-a-Service (BaaS), and/or simply as cloud computing platforms. As an example only, commercial cloud services such as Azure™, Supabase™ and/or Vercel™ can be used.

System 100 includes a least one user device 126, such as a smartphone, a laptop computer or a desktop computer, configured to operate application 124. Application 124 can for instance be served by an application server serving an application as SaaS 120 and be configured to generate a GUI in the form of a web page consisting of code in one or more computer languages, such as HTML, XML, CSS, JavaScript and ECMAScript. In some embodiments, the GUI can be generated programmatically, for instance on a server hosting application 124, and rendered by an application such as a web browser on the user device 126. In other embodiments, the application 124 can be configured to generate the GUI via a native application running on the user device 126, for example comprising graphical widgets configured to render information received from SaaS 120. The user device 126 is configured to allow the user to retrieve an audio file including the recording of the meeting between a professional and a person and to upload the audio file to the SaaS 120 via the application 124. In some embodiments, the user device 126 includes a sound sensor such as a microphone and is configured to record the meeting and to generate the audio file that is uploaded to the SaaS 120.

System 100 can include a BaaS 110 configured to offer storage service for the application 124 and/or for the infrastructure the application 124 relies on. BaaS 110 can for instance include a BaaS API 111, which provides an interface for application 124 and/or other services to create, query and retrieve data stored in BaaS 110. BaaS 110 can be configured to store some or all data in a relational database system 112. It can be appreciated that certain types of data, such as binary data like audio, can be advantageously stored in different types of storage, though, such as cloud storage containers 113, which can be provided by BaaS 110.

System 100 can include a cloud computing platform 150 configured to execute an orchestrator 152 that automates the deployment, management, and coordination of multiple services, applications, or containers within a cloud environment to ensure their seamless and efficient operation. The orchestrator 152 runs at least one serverless computing service 154 that provides an interface, for instance a RESTful interface allowing exchanges of data between SaaS 120 and platform 150. Once the application 124 receives an audio file of a meeting and the instruction to generate a document based on this file from a user device 126, an audio file upload 115 function of the PaaS can be used to upload the audio file to an audio storage 156 module of platform 150, and the application 124 can connect to the computing servive 154 to initiate the document generation workflow.

System 100 can include another cloud computing platform 140 including scalable transcription containers 148 which can deploy any number of transcriptor containers 144 such that platform 140 is configured to scale to allow for the transcription of any quantity of audio data. Platform 150 can be configured to transmit the audio file stored in audio storage 156 on to platform 140, where the meeting is transcripted. The transcription of the meeting can be sent to application 124, which can for instance cause the transcription to be displayed on user device 126, to be used as part of a prompt that is transmitted to platform 150, and/or to be transmitted to BaaS 110 for storage.

Cloud computing platform 150 can include a large language model 170 trained to accept the prompt received from application 124 as input and to generate a document as output. The generated document can be sent back to application 124, for instance so that it can be served to the user device 126 or stored in BaaS 110, and/or directly sent to BaaS 110 by platform 150.

System 100 can further include a cloud computing platform 190 configured to implement anonymization and/or pseudonymization services. Platform 190 can for instance implement an anonymization model 192 configured to detect personal, private, sensitive and/or protected information. Platform 190 can further implement an audio anonymization module 194 configured to anonymize audio data based on the output of the anonymization model 192. Information that is deemed sensitive and has to be removed or replaced can depend based on regulations enforceable in the location where system 100 is used and/or where the user is located. Sensitive information can include for instance the name of the person, all dates, including for instance date of birth of the person and date of the meeting, addresses, postal codes, organization names such as workplaces or companies, driver's license numbers, phone numbers, ages, social insurance number, email, health insurance information such as insured number, records number such as medical records number, medication, blood group, and/or illnesses, in particular rare or genetic diseases. In some embodiments, original transcriptions and/or meeting audio can be kept for a configurable length of time after being recorded and/or processed, for instance 48 hours, and then is removed or replaced with anonymized or pseudonymized transcriptions and/or meeting audio.

The anonymization model 192 can be implemented as part of a named entity recognizer (NER), which is configured to recognize named entities in meeting transcriptions, and in particular named entities corresponding to potentially sensitive information. Model 192 can for instance be a language model such as a bidirectional transformer language model trained specifically for and on transcriptions of meetings within system 100, or can be an off-the-shelf model, for instance Azure™ AI Language, Spark-NLP™, SpaCy, and/or GLINER, as described for instance in Zaratiana, Urchade, et al.; “Gliner: Generalist model for named entity recognition using bidirectional transformer”; arXiv preprint arXiv:2311.08526 (2023), the entire disclosure of which is hereby incorporated in its entirety, which has been configured to recognize named entities corresponding to sensitive information and/or whose output is analyzed to determine which recognized named entities correspond to sensitive information. In some embodiments, the model 192 is trained or configured so as to maximize a F_{β} score, with β>1, indicating that recall is more important than precision, i.e., that false negatives (missed sensitive information) are less desirable than false positives (information incorrectly characterized as sensitive). In other words, the model 192 can be trained or configured such that it is allowed to incorrectly recognize sensitive information in order to avoid incorrectly missing sensitive information.

In some embodiments, transcriptions are anonymized by removing any substring corresponding to a recognized named entity that corresponds to sensitive information, thereby generating an anonymized transcription, which can securely and/or legally be stored. In some embodiments, transcriptions are pseudonymized by replacing any substring corresponding to a recognized named entity that corresponds to sensitive information with a replacement substring, thereby generating a pseudonymized transcription, which can securely and/or legally be stored. In some embodiments, replacement substrings are selected randomly from named entities of the same class, such as named entities can be replaced with distinct named entities, e.g., a drug name will be replaced with a different drug name. In some embodiments, replacement substrings are selected randomly from named entities of the same class used in a similar context, e.g., the name a drug for a cardiac condition will be replaced with the name of a different drug for a cardiac condition.

Platform 190 can include an audio anonymization module 194 configured to anonymize audio data. In embodiments including an audio anonymization module 194, the transcription platform 140 can be configured to generate, in addition or as an alternative to the normal, or plain transcription of meetings, a verbose transcription, such as a timestamped transcription including timestamps. Using the verbose transcription and the list of named entities that correspond to sensitive information as input, the audio anonymization module 194 is configured to identify an initial timestamp, corresponding to the time at which or before which the sensitive information starts to be spoken in the audio data, and a terminal timestamp, corresponding to the time at which or after which the sensitive information ceases to be spoken in the audio data, and to crop the audio segments between the initial timestamp and the terminal timestamp out of the audio data, thereby generating an anonymized meeting audio, which can securely and/or legally be stored.

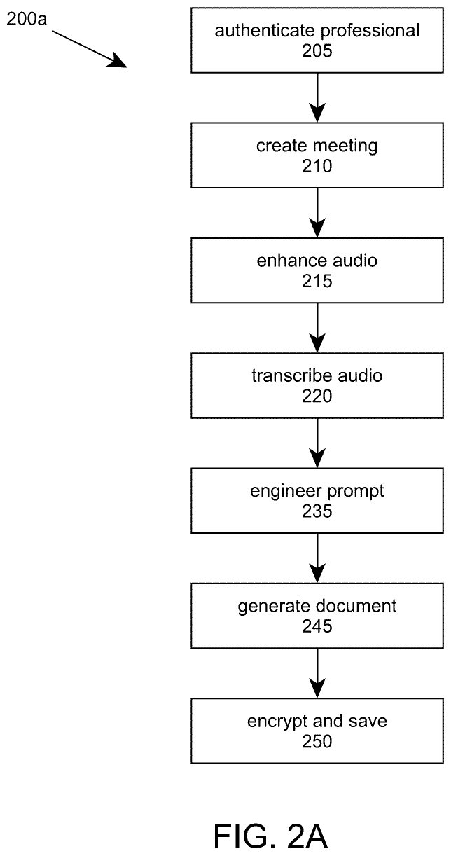

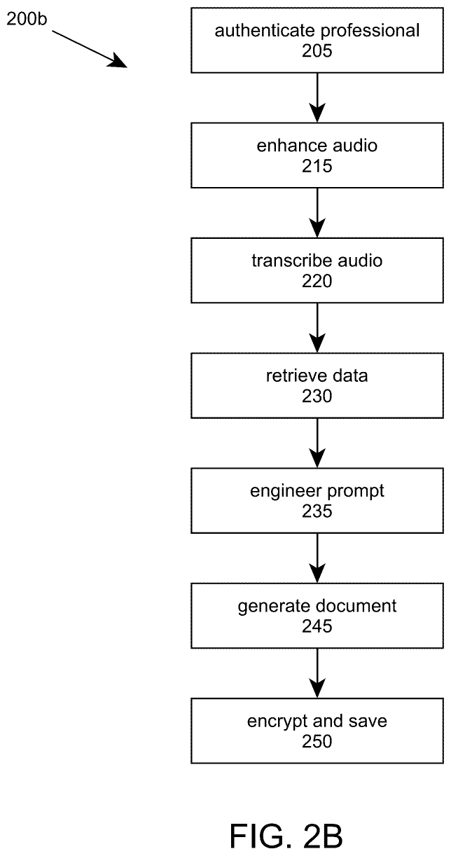

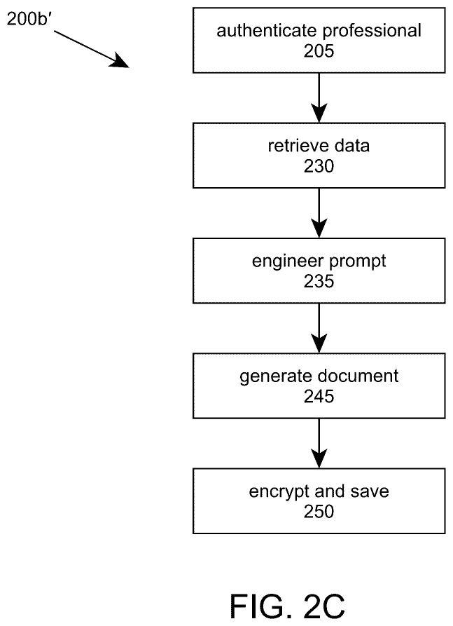

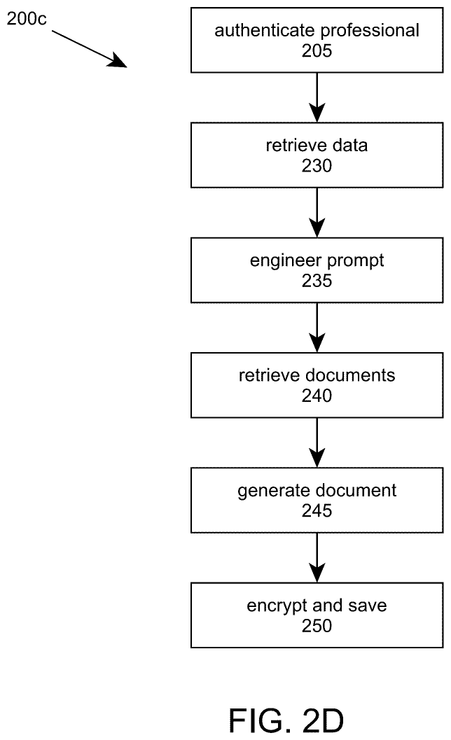

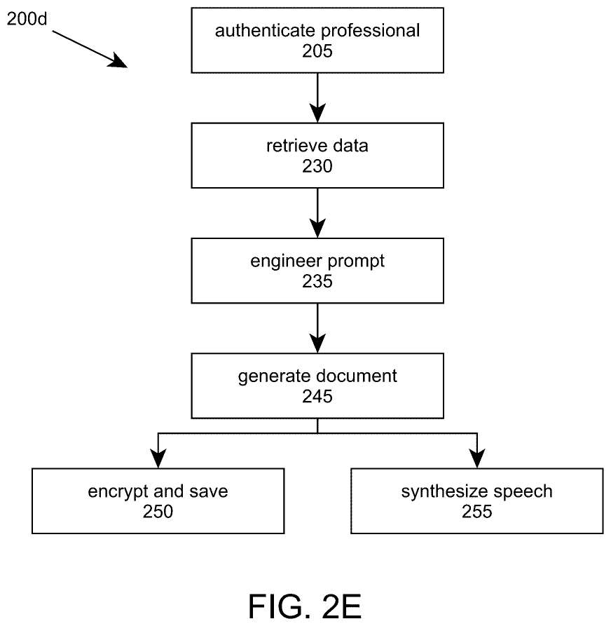

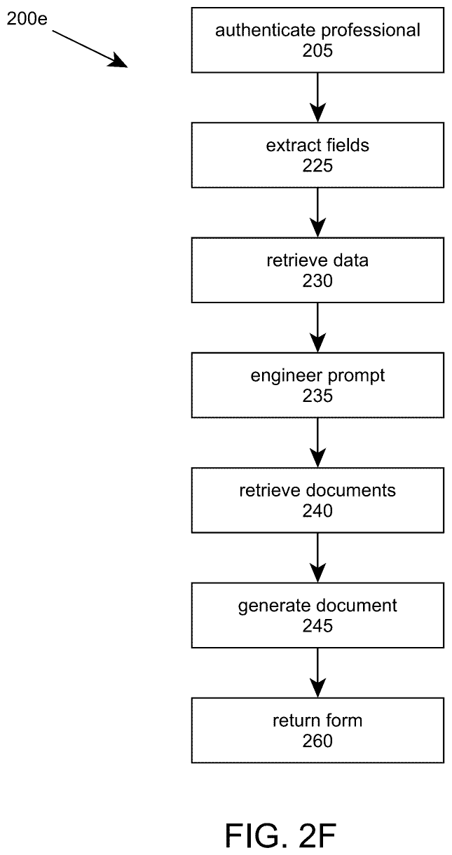

With reference to FIGS. 2A to 2F, exemplary methods for generating medical documents from audio data and/or from previously generated medical documents are shown. It can be appreciated that the methods can for instance be implemented using a system for generating personal documentation from audio data such as the system 100.

Certain steps are equivalent or analogous between two or more methods, as indicated for instance by the same reference numeral being used. It will be appreciated that a same step in two methods can be performed in the same way unless otherwise indicated.

With reference to FIG. 2A, an exemplary method 200a for generating a medical meeting summary from audio data is shown. Broadly described, method 200a allows a health professional to record a meeting with a patient and automatically generate a summary of said meeting.

The first step 205 includes the user of the system, i.e., the health professional, authenticating in the system. Any suitable authentication means can be used, such as for instance the user entering a username and a password and/or providing biometrics such as fingerprints and/or an image of their face for comparison with corresponding stored data.

A subsequent step 210 includes the user creating a meeting. For instance, this can include selecting a patient from a list of known patients, or creating a new patient profile. Once the meeting is about to start, the user can provide an input, for instance push a button on a user interface, to start the recording. Alternatively, the user can record the meeting using an application or device independent of the system. In that case, step 210 can include obtaining the audio recording from the storage means where it can be found.

A subsequent step 215 includes enhancing the recorded audio to improve the ease of obtaining a high quality transcription and/or to diminish the computational cost of automatically generating such a transcription. Step 215 can include reducing background noise, identifying and removing silent audio segments, and accelerating the audio.

A subsequent step 220 includes transcribing the recorded audio to obtain a transcription. The transcription can correspond to a transcript of what was said by the health professional and the patient during the meeting. The transcription can be augmented with annotations, for instance indicating who is talking and whether and which emotion is being expressed at each time throughout the meeting.

A subsequent step 235 includes generating a prompt, also called “engineering” a prompt, which is a text string including the transcription of the meeting and additional text generated so as to direct a generative language model to generate the summary in the desired format, with the desired structure and style, including for instance persona indications and examples of the desired output.

A subsequent step 245 includes providing the prompt as input to the generative language model and receiving the summary as its output. In some embodiments, the generative language model is structured such that it requires a different type of object as input, such as for instance a vector of integers. Step 245 can therefore include transforming the prompt to the appropriate type of object, for instance by using a tokenizer configured to take as input a text string such as a prompt and an encoding and to provide as output a vector of integers, each dimension of the vector corresponding to a character, a portion of a word or a word of the text string.

A final step 250 includes encrypting the summary generated at step 245 for secure storage.

With reference to FIG. 2B, an exemplary method 200b for generating a medical note from a summary and audio data is shown. Broadly described, method 200b allows a health professional to record a professional statement following a meeting with a patient and, using a previously generated summary of said meeting, automatically generate a note.

First steps 205, 215 and 220 include authenticating the health professional, enhancing the audio recording of the professional statement and transcribing said audio recording.

A subsequent step 230 includes retrieving additional data related to the meeting. In particular, this includes retrieving a summary of the meeting that was previously generated and stored, for instance by method 200a.

A subsequent step 235 includes engineering a prompt from the transcription and the additional data. A subsequent step 245 includes providing the prompt as input to the generative language model and receiving the note as its output. A final step 250 includes encrypting the note generated at step 245 for secure storage.

With reference to FIG. 2C, an exemplary method 200b′ for generating a medical note from a summary and a professional statement. Broadly described, method 200b′ allows a health professional to use a previously generated summary of a meeting with a patient and a written or transcribed professional statement to automatically generate a note.

The first step 205 includes authenticating the user.

A subsequent step 230 includes retrieving data related to a meeting with a patient. In particular, this includes retrieving a summary of the meeting that was previously generated and stored, for instance by method 200a, as well as a professional statement that was input by the user.

Subsequent steps 235, 245 and 250 include engineering a prompt from the data, providing the prompt as input to the generative language model and receiving the note as its output, and encrypting the generated note for secure storage.

With reference to FIG. 2D, an exemplary method 200c for generating a medical recommendation from one or more previously generated documents. Broadly described, method 200c allows a health professional to use a previously generated summary of a meeting with a patient, a written or transcribed professional statement and/or a previously generated note to automatically generate a medical recommendation. Method 200c can be referred to as the “Care Path” method, since generating recommendations along with the required medical records supports the patient in regularly improving their health and well-being.

The first step 205 includes authenticating the user.

A subsequent step 230 includes retrieving data related to a meeting with a patient. In particular, this includes retrieving a summary of the meeting that was previously generated and stored, for instance by method 200a, a professional statement that was input by the user, and/or a medical note that was previously generated and stored, for instance by method 200b or 200b′. In some embodiments, the data include patient data taken from the patient's profile and/or accessible records.

A subsequent step 235 includes engineering a prompt from the data.

A subsequent step 240 includes retrieving previously generated medical recommendations, portions of previously generated medical recommendations, or vectorial embeddings corresponding to previously generated medical recommendations or portions of previously generated medical recommendations. This can include executing a search algorithm to retrieve vectorial embeddings that are most similar to a vectorial embedding corresponding to the prompt and/or vectorial embeddings that are associated with patient data that are most similar to the patient data obtained in step 230. Step 240 furthermore includes augmenting the prompt generated in step 235 with the retrieved previously generated medical recommendations, portions of previously generated medical recommendations, or vectorial embeddings.

Subsequent steps 245 and 250 include providing the augmented prompt as input to the generative language model and receiving the recommendation as its output, and encrypting the generated recommendation for secure storage.

With reference to FIG. 2E, an exemplary method 200d for generating a medical case history from medical notes is shown. Broadly described, method 200d allows a health professional to use a configurable number of previously generated notes corresponding to the most recent meetings with a patient to automatically generate a medical case history.

The first step 205 includes authenticating the user.

A subsequent step 230 includes retrieving a configurable number, e.g., 5, of medical notes that was previously generated and stored, for instance by method 200b or 200b′, following the corresponding number of most recent meetings with a patient.

Subsequent steps 235, 245 and 250 include engineering a prompt from the notes, providing the prompt as input to the generative language model and receiving the case history as its output, and encrypting the generated case history for secure storage.

An additional step 255, which can be performed before, during or after step 250, include providing the generated case history as input to a speech synthesizer and performing a play back of the synthesized speech.

With reference to FIG. 2F, an exemplary method 200e for filling a form from medical data is shown. Broadly described, method 200e allows a health professional to use previously stored medical data to automatically fill a form. The form can be provided by the health professional or the patient, or can be one of a number of forms available through the system.

The first step 205 includes authenticating the user.

A subsequent step 225 includes extracting fields that need to be filled from the form. As an example, this can include retrieving fields and field captions from an XFA™ form, e.g., from a Portable Document Format (PDF) document, input elements and/or labels, e.g., from an HTML form, and/or fields and/or field names from an XFDF form, e.g., from a PDF document.

A subsequent step 230 includes retrieving data related to all previous meetings with a patient. In particular, this includes retrieving one or more meeting summaries that were previously generated and stored, for instance by method 200a, one or more professional statements that were input by the user, one or more medical notes that were previously generated and stored, for instance by method 200b or 200b′, and/or one or more medical recommendations that were previously generated and stored, for instance by method 200c. In some embodiments, the data include patient data taken from the patient's profile and/or accessible records.

Subsequent steps 235, 240 and 250 include, individually for each extracted field, engineering a prompt from the data and the unfilled form and retrieving previously generated filled fields, portions of previously generated filled fields, or vectorial embeddings corresponding to previously generated filled fields or portions of previously generated filled fields, augmenting the prompt therewith, and providing the augmented prompt as input to the generative language model and receiving each filled field as output. In some embodiments, only one prompt can be engineered for an entire form, reducing the number of model invocations necessary. In some embodiments, prompts can be generated for subsets of the form fields, in order to reach a tradeoff between generation quality and usage of computational resources. As an example, in some embodiments, one prompt is engineered for all the free-text input fields, and one prompt is engineered for all the other input fields.

A final step 260 include updating the form with the generated filled fields and making it available to the user, for instance for further editing, saving and/or printing.

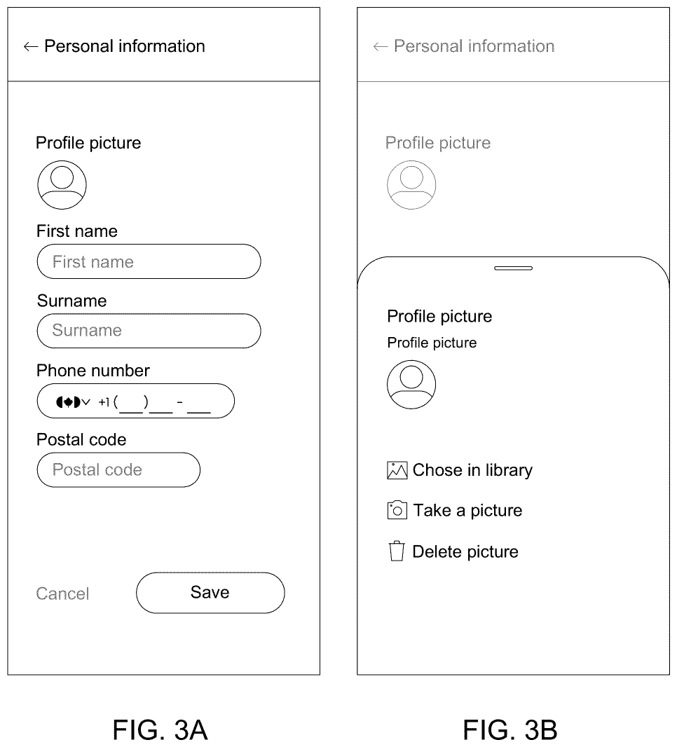

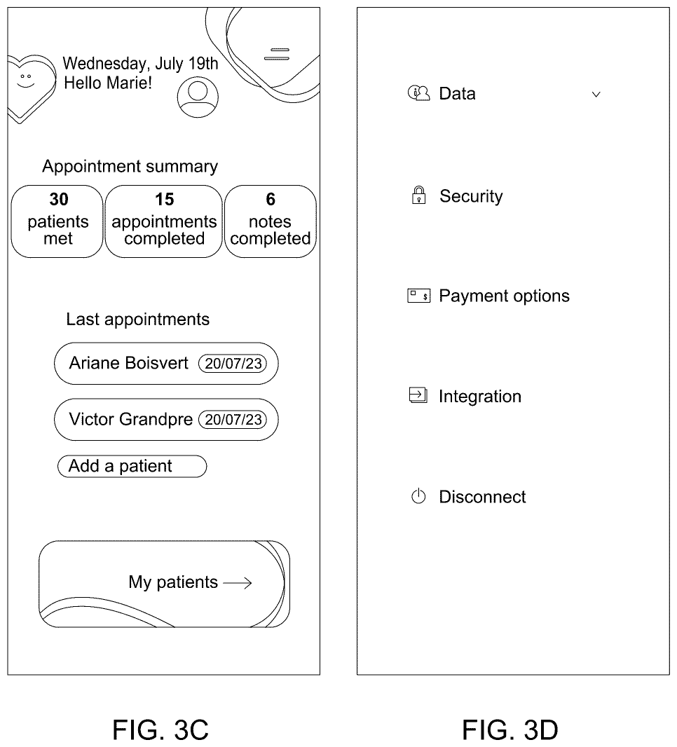

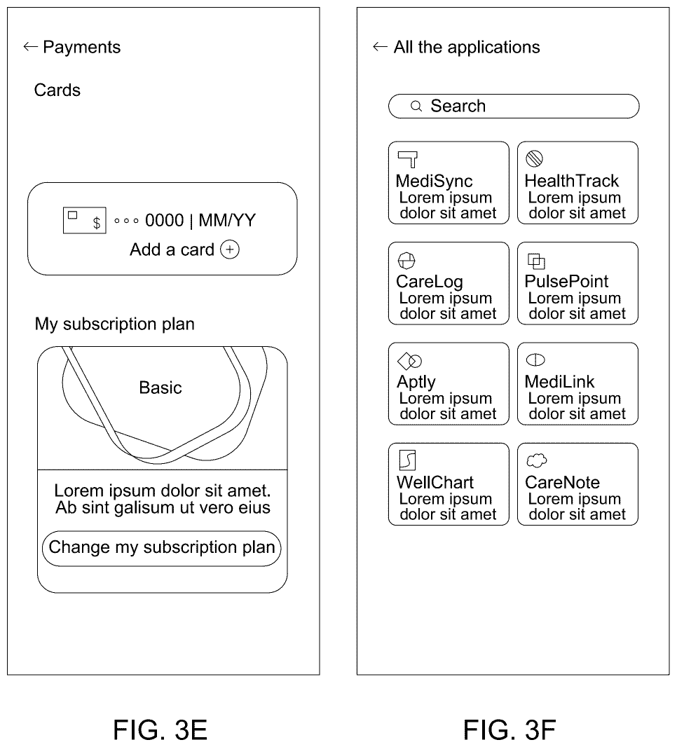

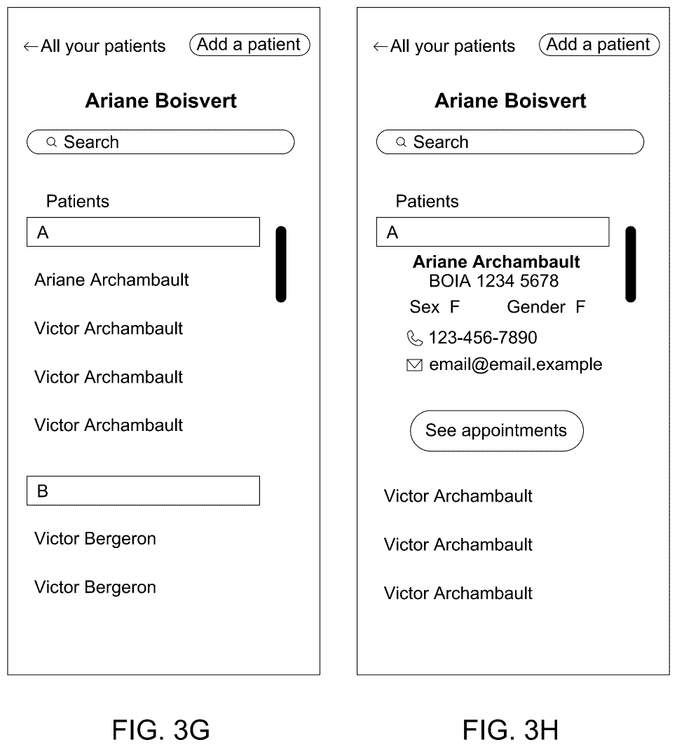

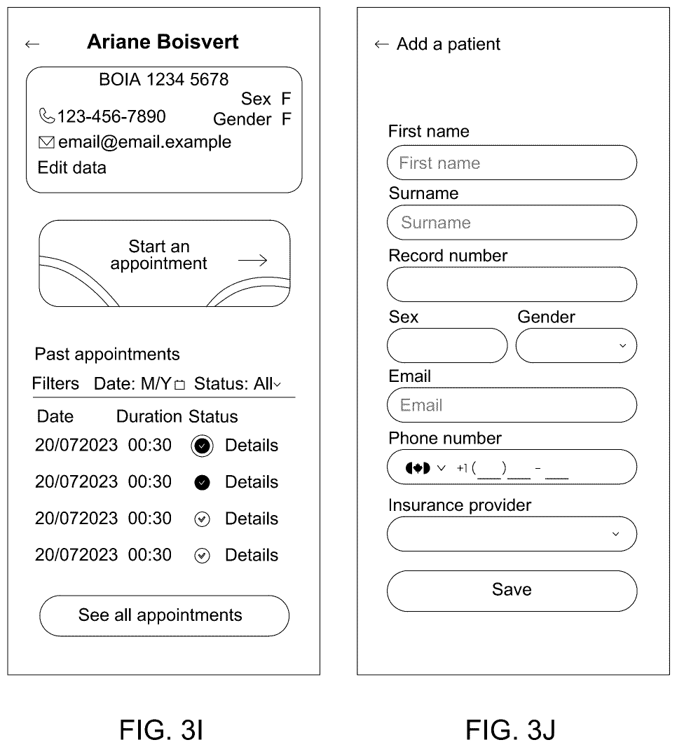

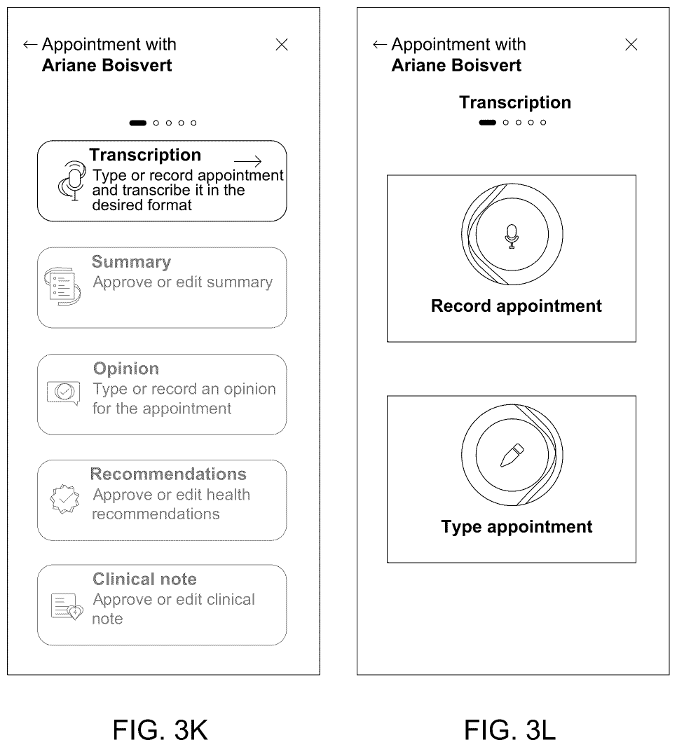

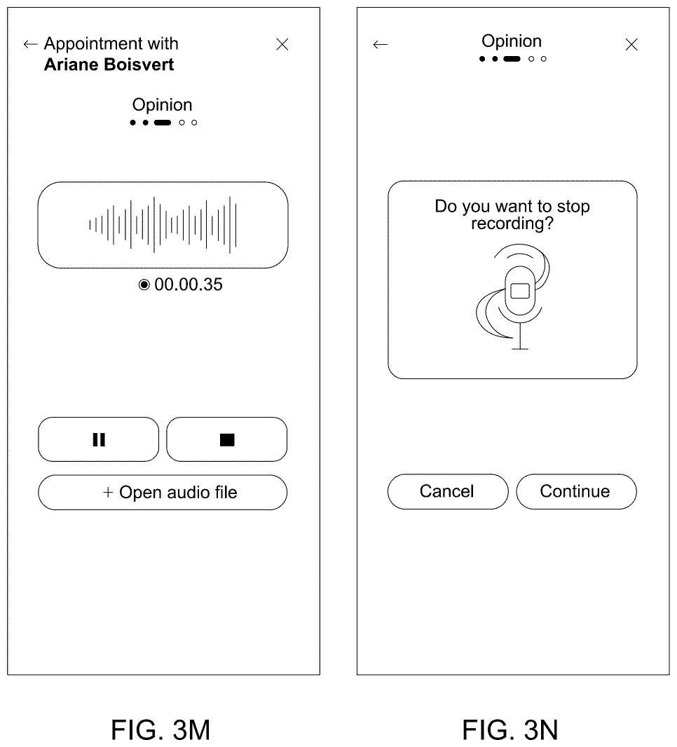

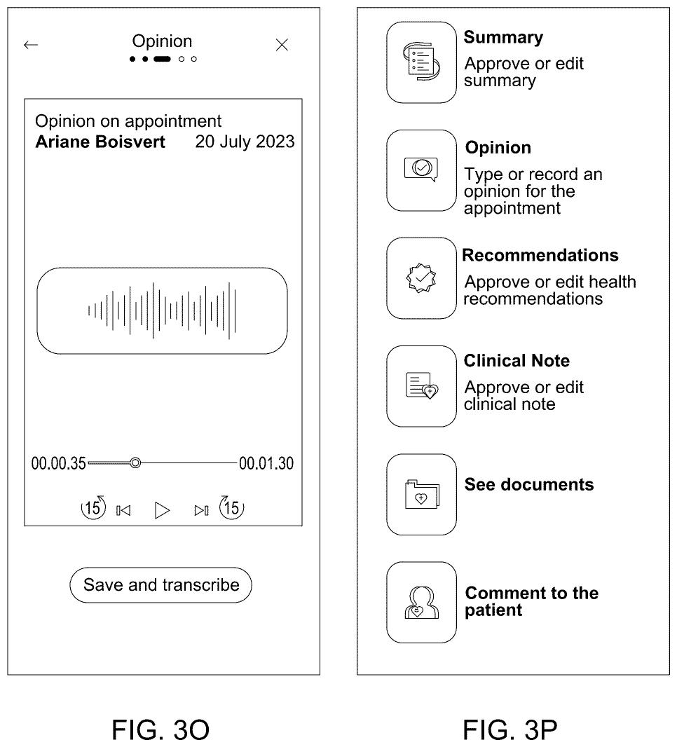

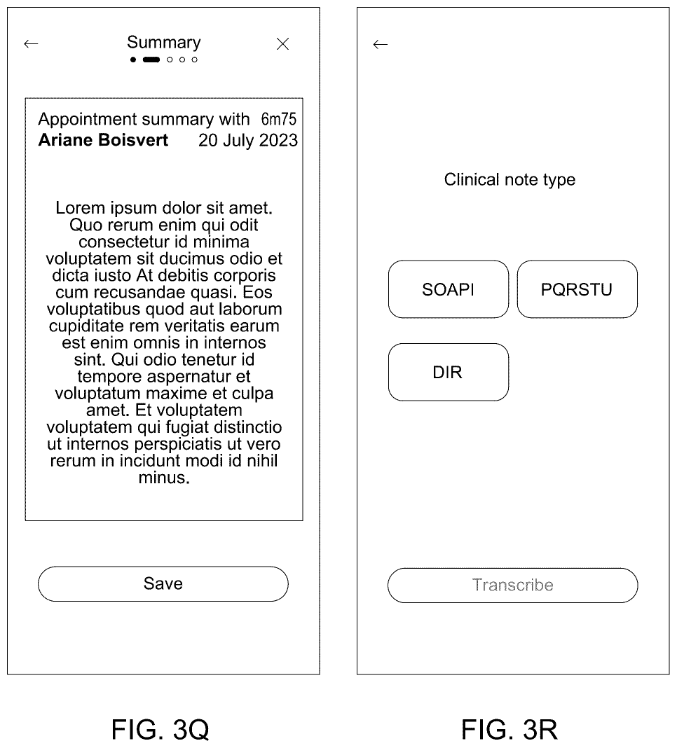

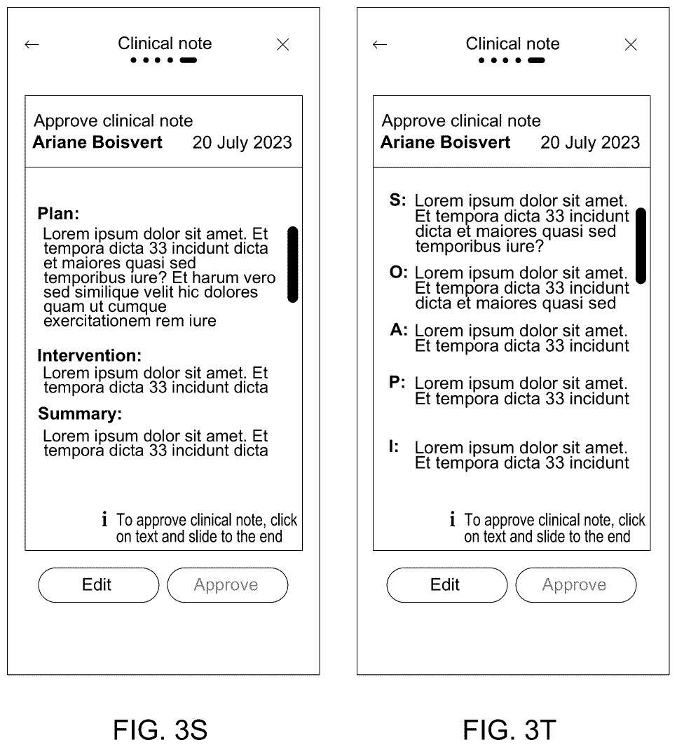

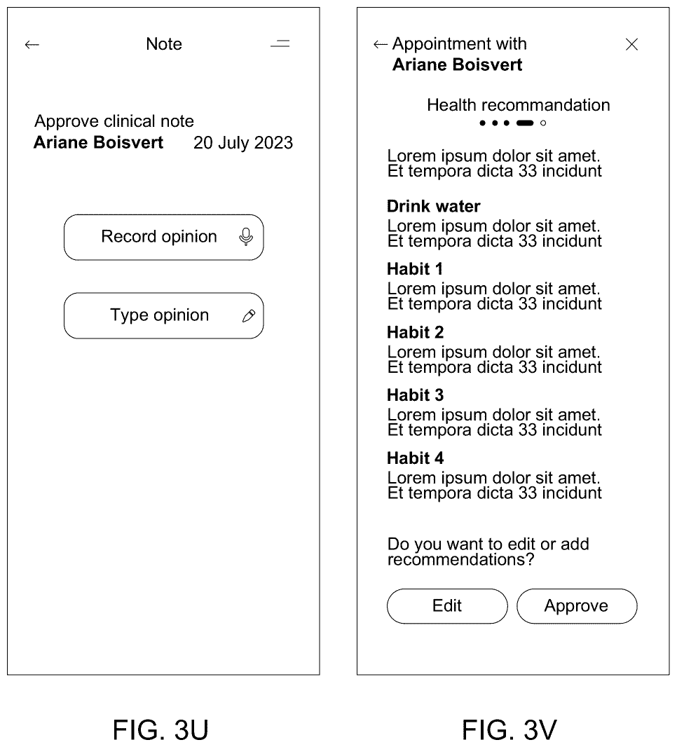

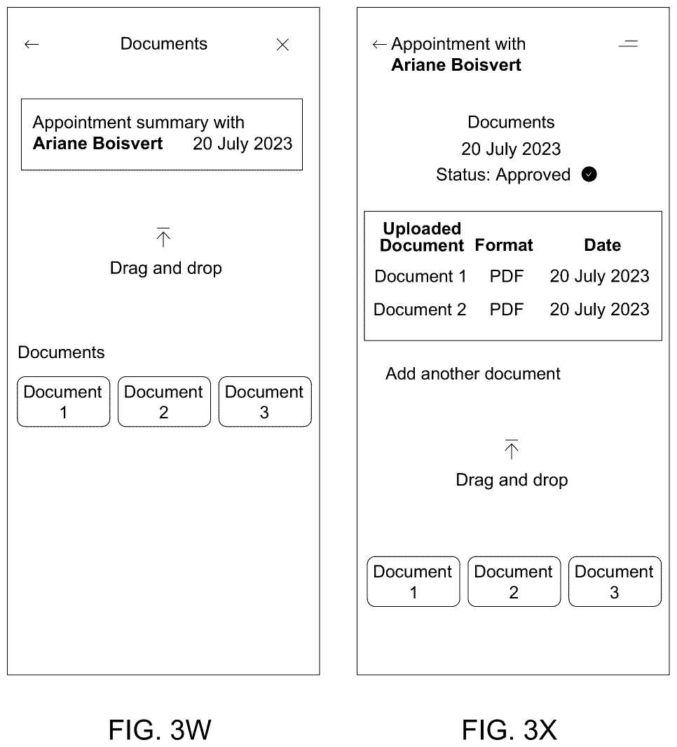

With reference to FIGS. 3A to 3Z, exemplary graphical interfaces for generating medical documents from audio data and/or from previously generated medical documents are shown. The shown graphical interfaces can for instance correspond to different views of the control centre 124 from FIG. 1A.

FIG. 3A shows a view of a screen allowing a professional to create or edit their user profile after authenticating.

FIG. 3B shows a view of a screen allowing the professional to select a photograph for their user profile.

FIG. 3C shows a view of a dashboard screen displayed to the professional after authenticating.

FIG. 3D shows a view of a preference screen displayed to the professional upon request.

FIG. 3E shows a view of a subscription and payment screen displayed to the professional upon request in the context where the system is managed by a service provider and the professional subscribes to the service.

FIG. 3F shows a view of an application integration centre screen displayed to the professional upon request and allowing the management of interconnected applications.

FIG. 3G shows a view of a screen allowing the professional to browse through their patients. Patients can be selected to display the screen of FIG. 3H.

FIG. 3H shows a view of a screen displayed to the professional upon selecting a patient from the list. The screen includes a button to display the screen of FIG. 3I.

FIG. 3I shows a view of a screen allowing the professional to see personal information related to the selected patient and details on the most recent meetings between the professional and the patient.

FIG. 3J shows a view of a screen allowing the professional to create a new patient profile.

FIG. 3K shows a view of a screen allowing the professional to create a new meeting with a patient.

FIG. 3L shows a view of a screen allowing the professional to select between recording the meeting with the patient for automatic transcription or entering a transcription manually.

FIG. 3M shows a view of a screen shown to the professional during the recording of the professional statement following a meeting.

FIG. 3N shows a view of a screen shown to the professional when stopping the recording of the professional statement.

FIG. 3O shows a view of a screen shown to the professional when the recording of the professional statement is completed.

FIG. 3P shows a view of a screen allowing the professional to view, generate and/or edit various documents related to a meeting or to a patient.

FIG. 3Q shows a view of a screen displaying a generated meeting summary to the professional.

FIG. 3R shows a view of a screen allowing the professional to select a standard type of medical note to be generated. While the Subjective, Objective, Assessment, Plan, and Interventions (SOAPI), the Provocative/Palliative, Quality/Quantity, Region/Radiation, Severity, Timing/Treatment and Understanding (PQRSTU) and the Data, Intervention and Results (DIR) types are shown, it can be understood that any type of medical note can be generated.

FIG. 3S shows a view of a screen displaying a generated medical note to the professional.

FIG. 3T shows a view of a screen displaying a generated SOAPI note to the professional.

FIG. 3U shows a view of a screen offering to the professional an option to record or type a comment before approving a medical note.

FIG. 3V shows a view of a screen displaying a generated medical recommendation to the professional.

FIG. 3W shows a view of a screen allowing the professional to upload forms to be filled in relation with a meeting with a patient.

FIG. 3X shows a view of a screen the uploaded forms to the professional.

The methods and systems disclosed herein rely, at least in part, on prompt engineering to generate documents such as medical notes in the desired format from a transcript of an encounter between a professional and a person. It is desirable for the generated docment to contain consistently the important information in an appropriate format, without hallucination. Various prompt engineering techniques were attempted to achieve optimum results, i.e., precise documents in the desired format, without hallucination.

To evaluate the effectiveness of different embodiments the prompt engineering submodule taught herein, a dataset including transcriptions of medical encounters between a healthcare professional and a patient as well as corresponding clinicat notes that have been verified and validated by healthcare professionals in accordance with specific note formats was assembled. This dataset was used to compare the scores generated by various embodiments of the prompt engineering submodule to assess their accuracy. WanDB was used to run tests, retrieve metrics and analyze test results.

The metrics measured were BLEU (Bilingual Evaluation Understudy), ROUGE (Recall-Oriented Understudy for Gisting Evaluation) and BERTScore. BLEU compares the n-grams of the generated text with those of the reference text to assess accuracy. The more similar the n-grams of the generated text are to those of the reference, the higher the BLEU score is. Unlike BLEU, ROUGE focuses more on recall, i.e., the extent to which the elements of the reference are present in the generated text. There are several variants of ROUGE, including ROUGE-N, which evaluates n-grams, and ROUGE-L, which evaluates similarity based on the longest subsequences. The BERTScore metric uses sentence representations obtained by pre-trained models like BERT™ to calculate the semantic similarity between the generated text and the reference. This makes it possible to better capture the meaning of the text beyond simple word matching.

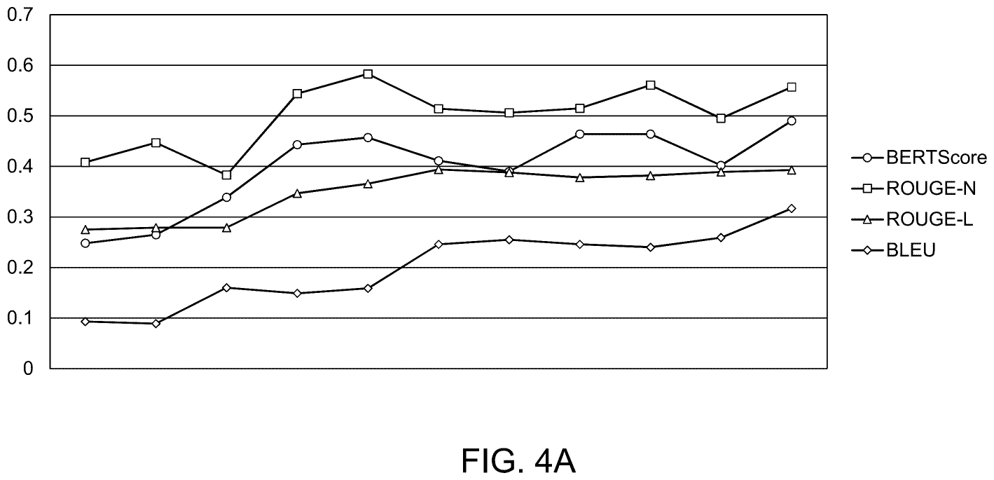

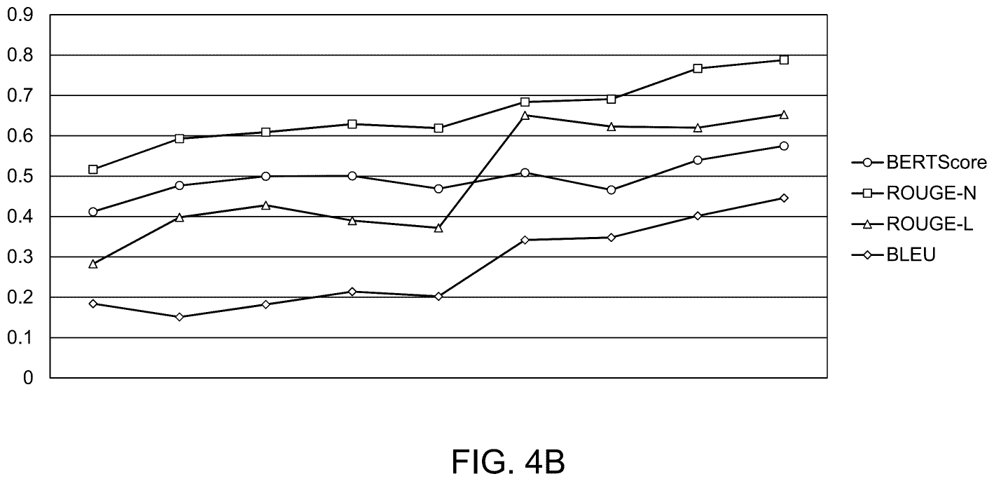

Two types of notes were generated based on different prompting techniques based on the transcripts from the dataset, using GPT-4o™ as the generative language model. The prompting techniques were tested incrementally in order to continually improve the cost/efficiency ratio with a fast generation time. FIGS. 4A and 4B show the progression over time of the BLEU score, the ROUGE-N F₁ score, the ROUGE-L F₁ score and the BERTScore F₁ score for each of the two types of noted evaluated.

In the first tests, notes were generated with a single invokation of the model. The desired note format and an example of transcription and generation were used in the prompt. The results were overall good, but sometimes crucial information was missing, which can be unacceptable in some applications. On GPT 3.5, the generation included omissions and was not exhaustive. With GPT-4, the performance gained was minimal, while generation time was increased.

Therefore, in subsequent tests, note formats were split into several parts, and a corresponding number of model invokations were performed, allowing the more to generate more detailed and accurate results by giving it simpler tasks, as described above. Although the results improved, the model showed a tendancy to generate undesired parts of a note. Moreover, including the transcript in each request required using more tokens, and therefore using more computational resources. Using one-shot prompting, i.e., adding an example improved the result, but also raised the number of tokens and therefore computational resources required.

Subsequent tests used Retrieval Augmented Generation (RAG) to improve our prompts. Transcript were split into several chunks of the same size, then each chunk was embedded to a vector and sent in a vector database. Based on the note format, the vector database was used to retrieve information relevant to each section, then retrieved short chunks containing information relevant to the section were used to generate the section. This makes it possible to instruct the model to find information based on short text excerpts rather than the complete transcript, diminishing the rate of error, hallucination and forgetfulness, providing more accurate and relevant answers, and importantly resulting in a smaller prompt size and fewer usage of computational resources. The prompt included context, an example, a format and a question to be answered. Unfortunately, the generations tended to include more information than necessary, resulting in repetitions between sections.

Subsequent tests revisited splitting the note in multiple parts for multiple invokations, with the model receiving the whole transcript at each invokation, but with an additional final invokation using the concatenated outputs in a prompt to check the format and content of the note and improve it if necessary, as described above. Unfortunately the model was not very proficient at performing this final task, and it would have become necessary to train a model for that specific end to improve results further.

Subsequent tests visited the idea of fine-tuning a pre-trained model, as described above. Since fine-tuning has to be performed separately for each type of document, the process would have proven too expensive computationally. Furthermore, a large amount of data would have been required to allow the model an adequate level of generality, requiring more human resources to assemble the data and more computational resources to fine-tube the model. Insufficient data, though, caused the model to hallucinate easily when an encounter was not very clear.

Numerous final tests were conducted using a single invokation of a general-purpose model, for improved computational efficiency, but with optimized prompts. Better organization of the personal, text structure and example indications, as described above, made it possible to achieve better results than the other techniques tested while maintaining required computational resources relatively low. In particular, prompts engineered by providing an indication a persona associated with a professional, a high-level indication of a text structure, an indication of a writing style, an indication of a tone, an indication of an intended audience, a low-level indication of a text structure and an example including an example transcript and an example document, in this order, provided the best results.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A system for generating personal documentation from audio data, the system comprising at least one processing device and at least one memory for storing instructions executable by the at least one processing device, the instructions implementing modules, the modules comprising:
an audio acquisition module configured to receive meeting audio of a meeting between a person and a professional and to store the meeting audio in the memory;
a transcription module configured to transcribe the meeting audio, generating a transcription and to store the transcription in the memory;
a text generation module configured to generate a document from at least the transcription, the text generation module comprising:
a prompt engineering submodule configured to generate a text string corresponding to a prompt from at least the transcription, and
a generative language model trained to take the text string as input and generate as output a continuation text string, wherein the continuation text string corresponds to a continuation of the text string predicted by the generative language model, and wherein the continuation text string further corresponds to the document; and
a storage module configured to securely store at least one of the meeting audio, the transcription and the document in a persistent storage device.

2. The system of claim 1, wherein the system is part of a electronic health records system, wherein the document is a medical document, the professional is a health professional and the person is a patient.

3. The system of claim 1, wherein the document is one of:
a meeting summary;
a meeting note;
a personal recommendation;
a filled form, wherein the prompt engineering submodule is configured to generate at least one field prompt from at least an unfilled form and the transcription; and
a case history.

4. The system of claim 1, wherein the text generation module is configured to:
generate an intermediary document, by the generative language model, from a first prompt based on at least the transcription, wherein the intermediary document corresponds to a compressed version of the first prompt; and
generate the document, by the same generative language model, from a second prompt based at least on the intermediary document and on an additional document.

5. The system of claim 1, wherein the text generation module is configured to:
generate a plurality of intermediary documents, by the generative language model, from a corresponding plurality of prompts, each prompt of the plurality based on at least the transcription, an indication of a document section and an indication of at least one example generated by an examples specifier, the indication of at least one example comprising textual instructions for the generative language model to rely on the at least one example in generating the document, each of the at least one example comprising at least an example transcription and an example section, each of the at least one example being selected from a set comprising predetermined examples, the plurality of intermediary documents corresponding to a plurality of document sections; and
generate the document by concatenating the plurality of intermediary documents.

6. The system of claim 1, wherein the prompt engineering submodule comprises a system instruction preparer configured to prepare system instructions for aggregation within the prompt, the system instructions comprising at least one of:
an indication of a persona generated by a persona specifier, the indication of a persona comprising textual instructions for the generative language model to adopt a persona corresponding to a scribe, to use a style based on a style of the professional, to use a professional tone, and to address an audience comprising peers of the professional;
an indication of text structure generated by a structure specifier, the indication of text structure comprising textual instructions for the generative language model to prepare a desired document type and to observe a desired text structure comprising a plurality of document sections, the indication of text structure selected based on the desired document type from a set comprising predetermined indications of text structure; and
an indication of at least one example generated by an examples specifier, the indication of at least one example comprising textual instructions for the generative language model to rely on the at least one example in generating the document, each of the at least one example comprising at least an example transcription and an example document, each of the at least one example being selected based on the desired document type from a set comprising predetermined examples.

7. The system of claim 6, further comprising a template builder configured to allow the professional to personalize the plurality of document sections, wherein the prompt engineering submodule is configured to select each of the at least one example based on the personalized document sections.

8. The system of claim 1, comprising a first, second, third and fourth remote cloud infrastructures, wherein:
the text generation module is implemented by the first remote cloud infrastructure;
the transcription module is implemented by the second remote cloud infrastructure;
the storage module implemented by the third remote cloud infrastructure; and
the first remote cloud infrastructure is in communication over a network with the second and third remote cloud infrastructures and with an application server, and is configured to:
receive the meeting audio from the application server,
transmit the meeting audio to the second remote cloud infrastructure for transcription and receive the transcription,
transmit the meeting audio and the transcription to the third remote cloud infrastructure for storage, and
generate and transmit the document to the application server.

9. The system of claim 1, further comprising a user interface, the user interface comprising:
an authentication module configured to authenticate a user of the system using credentials stored by the storage module; and
a control centre configured to allow an authenticated user to generate a document.

10. The system of claim 1, further comprising an anonymization module configured to generate an anonymized meeting audio and a pseudonymized transcription, wherein the transcription module is further configured to generate a timestamped transcription, the anonymization module comprising:
a named entity recognizer configured to recognize in the transcription named entities corresponding to private information;
a textual pseudonymization service configured to generate the pseudonymized transcription by replacing each of the recognized named entities with a distinct named entity of a same class; and
an audio anonymization service configured to generate the anonymized meeting audio by, for each named entity of the recognized named entities:
finding the named entity in the timestamped transcription,
retrieving an initial timestamp and a terminal timestamp corresponding to the named entity in the timestamped transcription, and
cropping an audio segment between the initial timestamp and the terminal timestamp out of the meeting audio.

11. A method for generating personal documentation from audio data, the method comprising:
receiving meeting audio of a meeting between a person and a professional;
transcribing the meeting audio, generating a transcription;
generating a text string corresponding to a prompt from at least the transcription;
generating a document from the prompt, by a generative language model trained to take the text string as input and generate as output a continuation text string, wherein the continuation text string corresponds to a continuation of the text string predicted by the generative language model, and wherein the continuation text string further corresponds to the document; and
securely storing at least one of the meeting audio, the transcription and the document in a distant persistent storage device.

12. The method of claim 11, implemented as part of a electronic health records system, wherein the document is a medical document, the professional is a health professional and the person is a patient.

13. The method of claim 11, wherein the document is one of:
a meeting summary;
a meeting note;
a personal recommendation;
a filled form, the method further comprising a step of generating at least one field prompt from at least an unfilled form and the transcription; and
a case history.

14. The method of claim 11, wherein generating the document comprises:
generating an intermediary document, by the generative language model, from a first prompt based on at least the transcription, wherein the intermediary document corresponds to a compressed version of the first prompt; and
generating the document, by the same generative language model, from a second prompt based at least on the intermediary document and on an additional document.

15. The method of claim 11, wherein generating the prompt comprises:
generate a plurality of intermediary documents, by the generative language model, from a corresponding plurality of prompts, each prompt of the plurality based on at least the transcription, an indication of a document section and an indication of at least one example generated by an examples specifier, the indication of at least one example comprising textual instructions for the generative language model to rely on the at least one example in generating the document, each of the at least one example comprising at least an example transcription and an example section, each of the at least one example being selected from a set comprising predetermined examples, the plurality of intermediary documents corresponding to a plurality of document sections; and
generate the document by concatenating the plurality of intermediary documents.

16. The method of claim 11, wherein generating the prompt comprises preparing system instructions for aggregation within the prompt, the system instructions comprising at least one of:
an indication of a persona comprising textual instructions for the generative language model to adopt a persona corresponding to a scribe, to use a style based on a style of the professional, to use a professional tone, and to address an audience comprising peers of the professional;
an indication of text structure comprising textual instructions for the generative language model to prepare a desired document type and to observe a desired text structure comprising a plurality of document sections, the indication of text structure selected based on the desired document type from a set comprising predetermined indications of text structure; and
an indication of at least one example comprising textual instructions for the generative language model to rely on the at least one example in generating the document, each of the at least one example comprising at least an example transcription and an example document, each of the at least one example being selected based on the desired document type from a set comprising predetermined examples.

17. The method of claim 16, further comprising allowing the professional to personalize the plurality of document sections, wherein generating the prompt comprises selecting each of the at least one example based on the personalized document sections.

18. The method of claim 11, further comprising:
authenticating a user of the system using stored credentials; and
allowing an authenticated user to generate a document.

19. The method of claim 11, wherein generating the transcription comprises generating a timestamped transcription, the method further comprising comprising:
recognizing in the transcription named entities corresponding to private information;
generating a pseudonymized transcription by replacing each of the recognized named entities with a distinct named entity of a same class; and
for each named entity of the recognized named entities:
finding the named entity in the timestamped transcription,
retrieving an initial timestamp and a terminal timestamp corresponding to the named entity in the timestamped transcription, and
generating an anonymized meeting audio by cropping an audio segment between the initial timestamp and the terminal timestamp out of the meeting audio.

20. A computer readable memory having recorded thereon statements and instructions for execution by a computer, said statements and instructions comprising:
receiving meeting audio of a meeting between a person and a professional;
transcribing the meeting audio, generating a corresponding transcription;
generating a text string corresponding to a prompt from at least the transcription;
generating a document from the prompt, by a generative language model trained to take the text string as input and generate as output a continuation text string, wherein the continuation text string corresponds to a continuation of the text string predicted by the generative language model, and wherein the continuation text string further corresponds to the document; and
securely storing at least one of the meeting audio, the transcription and the document in a distant persistent storage device.
